(19) United States
(12) Patent Application Publication
(10) Pub. No.: US 2023/0020297 A1
(43) Pub. Date: Jan. 19, 2023

(54) UOX-ALBUMIN CONJUGATE WITH CERTAIN NUMBERS OF ALBUMIN CONJUGATED THERETO, AND MANUFACTURING METHOD THEREOF

(71) Applicants: Proabtech Co., Ltd., Gwangju (KR); Gwangju Institute of Science and Technology, Gwangju (KR)

(72) Inventors: Jeong Haeng Cho, Gwangju (KR); Hyun Woo Kim, Gwangju (KR); Ja Yeon Lee, Gwangju (KR); lnchan Kwon, Gwangju (KR); Byungseop Yang, Gwangju (KR); Junyong Park, Gwangju (KR)

(21) Appl. No.: 17/297,850

(22) PCT Filed: Jun. 5, 2020
(86) PCT No.: PCT/KR2020/007328

§ 371 Date: May 27, 2021

(51) Int. Cl. A61K 47/64; A23L 33/195; A61P 13/12; A61P 19/06; C12N 9/06

(52) U.S. Cl. CPC A61K 47/64; A23L 33/195; A61P 13/12; A61P 19/06; C12N 9/0048; C12Y 107/03003

(57) ABSTRACT

The present application relates to a method of preparing urate oxidase (Uox) including a non-nature amino acid (NNAA) and Uox prepared thereby. The present application showed that the method of preparing Uox including an NNAA may be effectively used to prolong the half-life of a protein which is difficult to be linked to a carrier.

In addition, the Uox produced by the method may be effectively used for various biopharmaceuticals since its efficacy is maintained and drug persistency increases due to site-specific conjugation of a carrier, a risk of an immune response is reduced, and it is easily separated due to formation of uniform conjugate.

### RELATED APPLICATIONS

This application is a § 371 national-stage application based on PCT/KR2020/007328, filed Jun. 5, 2020 which is hereby incorporated by reference in its entirety.

### INCORPORATION BY REFERENCE OF SEQUENCE LISTING

The instant application contains a Sequence Listing which has been submitted electronically in ASCII format and is hereby incorporated by reference in its entirety. Said ASCII copy, created on Aug. 22, 2022, is named “PYH-00401_Sequence Listing” and is 15,248 bytes in size.

### TECHNICAL FIELD

The present application relates to a method of producing urate oxidase to which non-natural amino acids are introduced site-specifically and a process of producing an albumin conjugate thereof. An Uox-albumin conjugate can be prepared by conjugating an albumin using the non-natural amino acids.

In addition, the present application relates to a method of producing Uox, which is a tetramer formed by linkage of four monomers, and thus a specific number of albumins may be conjugated to the tetramer protein.

### Background Art

Over the last three decades, therapeutic proteins have attained clinical success in the treatment of various diseases, which continuously becomes a significant motivating factor in the pharmaceutical sector. One of the significant considerations for the development of a therapeutic protein is the extended duration of drug efficacy to avoid repeated injection. Since a therapeutic protein administered to a patient has been continuously removed from the patient's body, various methods for protecting the therapeutic protein from glomerulus filtration and an immune response have been tried.

The use of human serum albumin (HSA) as a carrier is one of the effective methods that can prolong the half-life of drugs in vivo. Specifically, HSA has a long half-life of 2 weeks or more in serum, and this seems to be due to the electrostatic repulsion in the kidney and an FcRn-mediated recycling action in the endothelium. The half-life of a therapeutic protein may be prolonged by conjugating the HSA to another protein using genetic incorporation or chemical bonding, which is referred to as albumination. In the case of a peptide or small-sized protein, its half-life was successfully increased and its pharmacodynamics (PD) was improved by albumination. However, in the case of a therapeutic protein having a multi-subunit and a complex, tertiary structure, it is not easy to obtain an albumin-mediated effect, which is due to an insufficient protein expression level and the considerable loss of protein activity caused by misfolding. There have been attempts to solve the problems of this general albumination method by site-specific protein conjugation. The flexible selection of a conjugation site and connection at a specific site show the possibility that a certain therapeutic protein can be conjugated to HSA regardless of a protein folding or multi-subunit problem.

Meanwhile, an extended genetic code brought about an innovative turning point in protein conjugation, and allowed site-specific incorporation of a non-natural amino acid (NNAA) into a target protein at any site of a target protein in all of various expression strains such as an E. coli, yeast or CHO cell line. Reactive NNAAs serve as a chemical handle, and allow molecules having functional groups with the same origin to be connected without a cross-reaction with other natural amino acids. The most noticeable thing is that this technique can be applied to site-specific PEGylation and various protein therapeutics such as an antibody-drug conjugate. When the NNAAs and the site-specific protein conjugation method are used together, since a therapeutic protein of interest can be made without loss of an innate function of the protein and it is possible to make manufacture process be efficient, the therapeutic proteins can be more effectively used clinically.

Thus, there is a need for a novel preparation method that prolongs the half-life of a therapeutic protein, and does not decrease its activity by site-specifically linking the NNAAs to the protein.

Therefore, the inventors confirmed that the use of fed-batch culture and three-step chromatography to prepare Uox containing albumin-conjugated NNAAs leads to obtaining Uox with a high yield, and specific mono-HSA-Uox and di-HSA-Uox suitable for treatment of a disease are provided in high purity, and thus the present application was completed.

### DISCLOSURE

### Technical Problem

The present application is directed to providing a method of preparing Uox containing NNAAs.

The present application is also directed to providing Uox according to the preparation method.

The present application is also directed to providing a pharmaceutical composition for preventing or treating gout, which includes Uox according to the preparation method.

The present application is also directed to providing a food composition for preventing or improving gout, which includes Uox according to the preparation method.

### Technical Solution

The present application provides a Uox-carrier conjugate of formula 1:

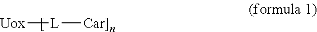

wherein UoX is a uricase from Aspergillus Flavus formed from 4 uricase monomers (p′);

L is a crosslinker; and

Car is a carrier,

wherein the p′ has an amino acid sequence of SEQ ID NO:1 substituted at at least one amino acid residue selected from the group of tyrosine 8, tyrosine 16, tyrosine 30, tyrosine 46, tyrosine 65, phenylalanine 79, phenylalanine 87, tyrosine 91, tryptophan 106, phenylalanine 120, phenylalanine 159, tryptophan 160, phenylalanine 162, tyrosine 167, tryptophan 174, tryptophan 186, tryptophan 188, phenylalanine 191, phenylalanine 204, tryptophan 208, phenylalanine 219, tyrosine 233, tyrosine 251, tyrosine 258, phenylalanine 259, tryptophan 265, and phenylalanine 279 by a non-natural amino acid selected from p-Azido-L-phenylalanine (AzF), p-ethynyl-phenylalanine (pEthF), L-Homopropargylglycine (HPG), O-propargyl-L-tyrosine (oPa) and p-propargyloxyphenylalanine (pPa),

the Uox and the L is linked through the substituted non-natural amino acid,

and n=2.

One exemplary embodiment of the present invention provides the Car is an albumin.

One exemplary embodiment of the present invention provides the p′ has an amino acid sequence of SEQ ID NO:1 substituted at tryptophan 160, or tryptophan 174 by a non-natural amino acid selected from p-Azido-L-phenylalanine (AzF), p-ethynyl-phenylalanine (pEthF), L-Homopropargylglycine (HPG), O-propargyl-L-tyrosine (oPa) and p-propargyloxyphenylalanine (pPa).

One exemplary embodiment of the present invention provides the substituted non-natural amino acid is p-Azido-L-phenylalanine (AzF).

One exemplary embodiment of the present invention provides the L comprises alkylene, alkenylene, alkynylene, or (CH2O)n.

One exemplary embodiment of the present invention provides the L comprises (CH2O)₁₀.

One exemplary embodiment of the present invention provides the albumin is a human serum albumin.

One exemplary embodiment of the present invention provides wherein the linked structure of the Uox and the L is a bonding structure formed by a click-chemistry reaction.

One exemplary embodiment of the present invention provides the linked structure of the Uox and the L is a bonding structure formed by a reaction between dibenzocyclooctyne (DBCO) and azide.

One exemplary embodiment of the present invention provides the linked structure of the L and the albumin is a bonding structure formed by a reaction between maleimide (MAL) and a cysteine residue of the albumin.

One exemplary embodiment of the present invention provides wherein the Uox is formed from two p′ each of which is not linked to the carrier and two p′ each of which is linked to the carrier.

To achieve the above object, according to one aspect of the present invention, there are provided a method for manufacturing the Uox-carrier conjugate, the method comprising:

producing a uricase comprising a non-natural amino acid by culturing bacteria by fed-batch culture;

separating the uricase comprising a non-natural amino acid;

conjugating the uricase comprising a non-natural amino acid with a carrier; and

separating the uricase conjugated to the carrier.

A method of preparing Uox including NNAAs according to the present application has excellent advantages in that Uox can be obtained with a high yield using fed-batch culture and three-step chromatography, and specific mono-HSA-Uox (single HSA-conjugated Uox) and di-HSA-Uox (double HSA-conjugated Uox), suitable for a disease, can be provided in high purity.

The method of preparing Uox, which includes NNAAs, according to the present application includes (1) culturing bacteria to produce Uox including NNAAs.

The “non-natural amino acid (NNAA)” used herein refers to an amino acid which is not one of the 20 common amino acids, pyrrolysine and selenocysteine, and other terms that can be used as a similar meaning to the term “non-natural amino acid (NNAA)” include “non-naturally encoded amino acid” and “non-naturally occurring amino acid”. The “non-natural amino acid (NNAA)” includes an amino acid which naturally occurs due to modification of a naturally-encoded NNAA, but is not introduced into a polypeptide grown by a translation complex, but the present invention is not limited thereto.

The Uox including NNAAs according to the present application may be prepared by substituting an amino acid residue selected from the group consisting of tyrosine at position 8, tyrosine at position 16, tyrosine at position 30, tyrosine at position 46, tyrosine at position 65, phenylalanine at position 79, phenylalanine at position 87, tyrosine at position 91, tryptophan at position 106, phenylalanine at position 120, phenylalanine at position 159, tryptophan at position 160, phenylalanine at position 162, tyrosine at position 167, tryptophan at position 174, tryptophan at position 186, tryptophan at position 188, phenylalanine at position 191, phenylalanine at position 204, tryptophan at position 208, phenylalanine at position 219, tyrosine at position 233, tyrosine at position 251, tyrosine at position 258, phenylalanine at position 259, tryptophan at position 265 and phenylalanine at position 279 of the amino acid sequence of SEQ ID NO: 1 with an NNAA such as p-azido-L-phenylalanine (AzF), p-ethynyl-phenylalanine (pEthF), L-homopropargylglycine (HPG), 0-propargyl-L-tyrosine (oPa) or p-propargyloxyphenylalanine (pPa). The p-azido-L-phenylalanine (Az) is a non-natural amino acid (NNAA) including an azide group.

Specifically, tryptophan at position 160, tryptophan at position 174 or tryptophan at position 160 and tryptophan at position 174 may be substituted with p-azido-L-phenylalanine (AzF). An amino acid sequence in which tryptophan at position 160 is substituted with p-azido-L-phenylalanine (AzF) is represented by SEQ ID NO: 2. An amino acid sequence in which tryptophan at position 174 is substituted with p-azido-L-phenylalanine (AzF) is represented by SEQ ID NO: 3. An amino acid sequence in which tryptophan at position 160 and tryptophan at position 174 are substituted with p-azido-L-phenylalanine (AzF) is represented by SEQ ID NO:4.

The amino acid sequence of SEQ ID NO: 1 may be a sequence encoded by a nucleotide sequence of SEQ ID NO: 5.

Although AzF is incorporated into Uox, it should affect the structure and function of the original Uox as little as possible, to determine an optimal site, aromatic amino acids (Phe, Trp and Tyr) structurally similar to AzF were selected as potential targets. Natural Uox has 10 phenylalanines, 7 tryptophans, and 10 tyrosines. In addition, a site with high solvent accessibility is more suitable for including AzF, this is because the higher the collision probability with a linker, the more effective the conjugation is. As a result of analyzing the solvent accessibility for 10 phenylalanines, 7 tryptophans and 10 tyrosines using an ASA-View server, four sites (W160, W174, F258 and W264) showed relatively high solvent accessibility. Among them, F258 and W264 were excluded since they are located at sites that are able to affect the formation of a multi-Uox subunit conjugate, and thus W160 and W174 showing a solvent accessibility of 0.46 and 0.62, respectively, were selected as sites for including AzF. W160 and W174 were determined as more suitable sites because it has not been reported that they play a critical role in the structure/function of Uox yet. The description of Uox including the NNAA is disclosed in the description related to Korean Patent Publication No. 10-1637010, and incorporated herein by reference.

The “urate oxidase (Uox)” used herein is an enzyme that oxidizes uric acid to produce allantoin and hydrogen peroxide, and is present in large amounts in the liver, kidney and the like of all mammals except primates. A human has the Uox gene, which is not activated, but does not have the Uox (protein), and therefore, the final metabolite of purine becomes uric acid. When there is an excessive concentration of uric acid in the blood, gout may be caused. Uox has the form of a tetramer composed of the same subunits, in which the same four active sites are located at the interface between the four subunits. According to an exemplary embodiment of the present application, Uox is derived from Aspergillus flavus, and each subunit is composed of 301 amino acids, and has a molecular weight of approximately 34 kDa.

In the present application, the bacteria are, for example, a strain selected from the genus Escherichia, the genus Erwinia, the genus Serratia, the genus Providencia, the genus Corynebacterium, the genus Pseudomonas, the genus Leptospira, the genus Salmonella, the genus Brevibacterium, the genus Hyphomonas, the genus Chromobacterium, the genus Nocardia, fungi or yeast. Preferably, the bacteria are an Escherichia coli strain.

More preferably, the expression vectors and production strains described in the prior patent (KR101637010) may be used. According to one embodiment, E. Coli C321.ΔA.exp [(pEVOL-AzF)(pQE80-Uox.W160,174amb)] may be used.

Preferably, the vector is preferably an expression vector prepared by removing six His residues from the C terminus of a conventional pQE80-Uox plasmid.

Specifically, the vector may include a pEVOL-pAzF plasmid (Plasmid ID: 31186) which includes tyrosyl-tRNA synthetase derived from Methanococcus jannaschii and an AzF-specific engineered pair consisting of amber suppressor tRNA.

To construct the expression vector by removing six His residues from the C terminus of a conventional pQE80-Uox plasmid, the following primers may be used:

F:
(SEQ ID NO: 6)
5-CTCTCTGAAGAGCAAGCTGTAAGCTTAATTAGCTGAGC-3
R:
(SEQ ID NO: 7)
5-GCTCAGCTAATTAAGCTTACAGCTTGCTCTTCAGAGAG-3

In addition, to substitute tryptophans at positions 160 and 174 of Uox with an amber codon (UAG), site-directed mutagenic PCR may be performed using the pQE80-Uox as a template, and to introduce the amber codon at position 160, 5-CAATTCACAGTTTTAGGGGTTTCTGAG-3 (SEQ ID NO: 8) and 5-CTCAGAAACCCCTAAAACTGTGAATTG-3 (SEQ ID NO: 9) primers may be used, and to introduce the amber codon at position 174, 5-CACTGAAGGAGACTTAGGATAGAATCCTG-3 (SEQ ID NO: 10) and 5-CAGGATTCTATCCTAAGTCTCCTTCAGTG-3 (SEQ ID NO: 11) primers may be used.

Accordingly, the vector according to the present application may include a nucleotide sequence of SEQ ID NO: 12.

That is, preferably, the bacteria according to the present application are E. Coli having pQE80-Uox.W160,174amb containing the nucleotide sequence of SEQ ID NO: 12 and a pEVOL-pAzF plasmid. More preferably, the bacteria according to the present application are E. Coli C321 having pQE80-Uox.W160,174amb containing the nucleotide sequence of SEQ ID NO: 12 and a pEVOL-pAzF plasmid. More preferably, the bacteria according to the present application are E. Coli C321 having pQE80-Uox.W160,174amb consisting of the nucleotide sequence of SEQ ID NO: 12 and a pEVOL-pAzF plasmid.

Uox including an NNAA comprising an amino acid sequence of SEQ ID NO: 4 according to the present application using the nucleotide sequence of SEQ ID NO: 12 may be provided.

In the present application, in a step of culturing the bacteria to produce Uox including an NNAA in Step (1), Uox may be produced using an expression vector. Compared to an expression vector of the conventional Uox having six His residues binding to the C-terminus for ease of separation and purification, six His residues are excluded from the expression vector in the present application. The conventional Uox expression vector with six His residues may cause unexpected side effects while being used commercially, whereas the expression vector according to the present application may not include a His-tag such that an enzyme suitable for human application and use can be provided.

In the present application, in Step (1) of culturing the bacteria to produce Uox including an NNAA, culturing may be performed according to fed-batch culture. Preferably, E. coli may be cultured through fed-batch culture. When E. coli cells proliferate to a certain level, growth may be inhibited due to increased pH or depletion of a carbon source, which is a nutrient in the medium. As this situation is left unattended, cell disruption occurs. As a medium concentration is able to be controlled to a constant level by using fed-batch culture which provides an additional medium at a constant rate to suitably maintain bacterial growth, a high concentration of bacteria may be grown by reducing the concentration of an organic acid, which is a by-product that inhibits growth, resulting in maximizing the culture efficiency of E. coli. That is, as the cells are grown by refilling the consumed medium by continuous replenishment of the medium, this method has a potential to achieving a high cell density according to a medium formulation, a cell line and other cell culture conditions. During cell culture, it is important to collect a large number of wet cells to ensure productivity, fed-batch culture may obtain 4-fold or more wet cells, compared to batch culture. Preferably, about 240 g/L of wet cells may be obtained by fed-batch culture.

The culture may be performed by main culture following two rounds of seed culture. The seed culture may be performed one to three times as needed.

Specifically, the medium may include, for example, the following medium conditions:

8 to 16 g/L, preferably 10 to 14 g/L, and more preferably, approximately 12 g/L of tryptone;

8 to 16 g/L, preferably 10 to 14 g/L, and more preferably, approximately 12 g/L of a yeast extract;

1.6 to 4.8 g/L, preferably 2.4 to 4.0 g/L, and more preferably, approximately 3.2 g/L of KH₂PO₄,

14 to 20 g/L, preferably 15.5 to 18.5 g/L, and more preferably, approximately 17.4 g/L of K₂HPO₄,

0.02 to 0.3 g/L, preferably 0.05 to 0.2 g/L, and more preferably, approximately 0.1 g/L of thiamine-HCl,

10 to 30 g/L, preferably 15 to 25 g/L, and more preferably, approximately 20 g/L of glucose, and

0.8 to 1.6 g/L, preferably 1.0 to 1.4 g/L, and more preferably, approximately 1.2 g/L of MgSO₄.

The medium may further include antibiotics, for example, ampicillin and chloramphenicol. Specifically, the medium may include 50 to 200 μg/mL, preferably, 75 to 150 μg/mL, and more preferably, approximately 100 μg/mL of ampicillin; and 10 to 60 μg/mL, preferably, 20 to 50 μg/mL, and more preferably, approximately 35 μg/mL of chloramphenicol.

After seed culture, the inoculation for culture may be performed with approximately 5 to 20%, preferably 10 to 15%, and more preferably, approximately 13.3% of the secondary seed culture liquid, and the culture may be performed under an oxygen supply condition at 28 to 32° C., and preferably 30° C.

When the concentration of the carbon or nitrogen source declines, an additional carbon source medium containing 300 to 900 g/L, preferably 500 to 700 g/L and more preferably, approximately 600 g/L of glucose and

0.8 to 1.6 g/L, preferably 1.0 to 1.4 g/L and more preferably, approximately 1.2 g/L of MgSO₄; and

an additional nitrogen source medium containing 80 to 160 g/L, preferably 100 to 140 g/L and more preferably, approximately 120 g/L of a yeast extract and

1.0 to 2.0 g/L, preferably 1.25 to 1.75 g/L and more preferably, approximately 1.5 g/L of ammonium sulfate may be added.

Here, the addition may be made in a pulse manner, and may be performed every 1 to 5 hours, and preferably 2 to 4 hours after approximately 10 hours.

When O.D. is approximately 10 or more, protein expression may be induced by additionally adding IPTG and arabinose while adding 0.5 to 10 mM, preferably 1 to 5 mM, and more preferably, approximately 3 mM of AzF.

Afterward, when the cells are grown so that the O.D. becomes approximately 190 to 220 or more, after centrifugation at 0 to 10° C., preferably 2 to 6° C. and more preferably 4° C. at 1000 to 10000 rpm, preferably 5000 to 7000 rpm and more preferably, approximately 6000 rpm, pellets obtained thereby may be stored at −100 to −60° C., preferably −90 to −70° C., and more preferably, approximately −80° C.

In the step of culturing the bacteria to produce Uox including an NNAA in Step (1) of the present application, the bacteria may be cultured at pH 6.5 to 7.5, and preferably approximately 7.0.

According to a preferable embodiment of the present application, fed-batch culture is performed under the following conditions:

The cells were cultured under continuous oxygen supply at 30° C., pH 7.0, 600 rpm, 1 vvm, and 100% DO.

Primary seed culture is performed at 37° C. and 200 rpm for 15 hours after 0.75% inoculation into a medium containing 12 g/L tryptone, 24 g/L yeast extract, 5 g/L glycerol, 2.3 g/L KH₂PO₄, 12.53 g/L K₂HPO₄, 100 μg/mL ampicillin and 35 μg/mL chloramphenicol.

Secondary seed culture is performed with 5% inoculation of the primary seed culture liquid to reach an O.D. of 5 or more at 30° C., 500 rpm and 1 vvm.

Main culture is performed with 13.3% inoculation of the secondary seed culture liquid in a medium containing 12 g/L tryptone, 24 g/L yeast extract, 3.2 g/L KH₂PO₄, 17.4 g/L K₂HPO₄, 100 μg/mL ampicillin, 35 μg/mL chloramphenicol and 0.1 g/L thiamine at 30° C., pH 7.0, 600 rpm, 1 vvm and 100% DO.

When the O.D. is 10 or more, protein expression is induced by adding 3 mM AzF, 1 mM IPTG and 0.2% arabinose.

At the time when the concentration of the carbon source declines, cell growth is induced by providing an additional carbon source medium containing 600 g/L of glucose and 1.2 g/L of MgSO₄, and an additional nitrogen source medium containing 240 g/L of a yeast extract and 1.5 g/L of ammonium sulfate at constant rates.

After the end of the culture, pellets are obtained by centrifugation at 4° C. and 6000 rpm for 10 minutes, and stored at −80° C.

According to one embodiment of the present application, the step of culturing bacteria to produce Uox including an NNAA is performed as follows:

E. Coli C321 transformed with a pQE80-Uox.W160amb, pQE80-Uox.W174amb, and/or Uox.W160,174amb plasmid(s) containing a nucleotide sequence of SEQ ID NO: 12, and a pEVOL-pAzF plasmid is cultured by fed-batch culture, and thereby, Uox including an NNAA at position(s) 160 and/or 174 of Uox may be produced by substituting tryptophan(s) 160 and/or 174 of Uox (based on SEQ ID NO: 1) with an NNAA such as p-azido-L-phenylalanine (AzF).

The method for manufacturing Uox including an NNAA according to the present invention includes separating the Uox including an NNAA (Step (2)).

In the present application, the step of separating the Uox (2) may be performed by 2-step, and preferably, 3-step or more chromatography. Specifically, the separation of the Uox may be accomplished by sequentially performing hydrophobic chromatography, anion exchange chromatography and size exclusion chromatography. In addition, specifically, the separation of Uox is accomplished by sequentially performing anion exchange chromatography, hydrophobic chromatography and size exclusion chromatography.

In the present application, by sequentially performing hydrophobic chromatography, anion exchange chromatography and size exclusion chromatography, a yield may increase, and an enzyme suitable for producing a specific Uox-albumin conjugate such as mono-HSA-Uox or di-HSA-Uox may be provided. More preferably, in the present application, the anion exchange chromatography, the hydrophobic chromatography and the size exclusion chromatography may be sequentially performed.

In the present application, by further performing size exclusion chromatography, high-purity Uox, and preferably, Uox in a purity of 95% or more may be obtained.

When the separated high-purity Uox is conjugated with a carrier, since Uox is contained at a higher purity and a higher content than a Uox-albumin conjugate used in conventional methods, it is possible to increase a production yield, and the conjugate is more suitable for use as a pharmaceutic.

Specifically, the anion exchange chromatography may be performed using an anion exchange resin selected from DEAE FF, Capto-Adhere, Bestarose Diamond MMA and Hitrap Q HP. Preferably, the chromatography is performed using a DEAE FF resin.

Specifically, the hydrophobic chromatography may be performed using a resin selected from phenyl sepharose HP, phenyl sepharose FF, phenyl bestarose HP, phenyl bestarose FF and Hitrap phenyl FF(HS). The hydrophobic chromatography is preferably performed using a Hitrap phenyl FF(HS) resin.

The “size exclusion chromatography (SEC)” used herein refers to a technique of separating a mixture based on a rate of passing various sizes of solutes through a porous matrix (transmittance). That is, the SEC utilizes a principle in which, when a sample for analysis passes through a column filled with a porous stationary phase such as a gel, a matrix or beads, large molecules that cannot pass through pores of the column may not enter pores and rapidly move through the column due to the surrounding empty space, whereas small molecules relatively slowly move through pores of the column. This method is generally used in desalting for buffer exchange, separation for purification or measurement of a molecular weight according to a solute size.

The most widely used gels for size exclusion chromatography are based on Sepharose (GE Healthcare), Superose (GE Healthcare), Sephadex (Pharmacia), Bio-Gel P (Bio-Rad), Superdex® (GE Healthcare) and TSKgel® (silica-based; Sigma), and according to one embodiment of the present application, Superdex 200 increase 10/300 GL may be used.

According to one embodiment of the present application, anion exchange chromatography, hydrophobic chromatography or size exclusion chromatography may be performed under following conditions:

In the anion exchange chromatography, an anion exchange column such as Hitrap DEAE FF is equilibrated with 20 mM Tris-HCl buffer (pH 9.0), and eluted by a NaCl gradient method. Afterward, separation is performed using a hydrophobic column such as Hitrap phenyl FF by mixing an active fraction and 20 mM Tris-HCl buffer (pH 9.0) containing 1M (NH₄)₂SO4 in 1:1.

For separation with more purity, size exclusion chromatography is performed. As a column, the Superdex 200 increase 10/300 GL is used.

According to one embodiment of the present application, the separation of the Uox including an NNAA (Step (2)) includes the following steps:

performing anion exchange chromatography using a Hitrap DEAE FF column;

performing hydrophobic chromatography using a Hitrap phenyl FF column; and

performing size exclusion chromatography using a Superdex 200 increase 10/300 GL column, wherein

the anion exchange chromatography, the hydrophobic chromatography and size exclusion chromatography are sequentially performed.

The method for manufacturing Uox including an NNAA according to the present application includes (3) conjugating the Uox including an NNAA with a carrier.

In the present application, the step of conjugating the Uox including an NNAA with a carrier (Step (3)) may be performed by mixing and reacting Uox and a carrier in a molar ratio of 1:1 to 1:5, preferably, 1:1 to 1:3, and specifically 1:1, 1:2 or 1:3.

Specifically, in the present application, the carrier is preferably albumin.

The “albumin” used herein refers to a protein which is most abundantly and widely distributed in the living body. Human albumin is a simple protein which consists of 585 amino acids, has a molecular weight of 66 kDa, and has 17 disulfide bonds and one free cysteine residue in a molecule. Albumin is made in the liver, secreted into the blood, and accounts for approximately 60% of proteins present in whole plasma, and as its physiological functions, (1) control and maintenance of plasma osmotic pressure, (2) transport of bilirubin, amino acids, fatty acids, hormones, metal ions, drugs, etc., (3) amino acid sources in malnutrition and (4) oxidation/reduction buffering capacity are known.

In the present application, an albumin conjugate is generated by conjugating the Uox including an NNAA with a carrier (Step (3)). The albumin conjugate refers to a material in which the Uox including an NNAA is conjugated with a carrier, and the present application is for obtaining an albumin conjugate in which the carrier is linked to a desired site of the Uox by reaction of the Uox with a carrier under specific conditions.

In Step (3) of conjugating Uox with a carrier according to the present application, the carrier may be site-specifically linked to the Uox.

The conjugate in which Uox is conjugated with a carrier (e.g., albumin) according to the present invention may be prepared by site-specifically conjugating a carrier, preferably, albumin, to Uox with a linker. Site-specific conjugation means conjugation of albumin at the position of a specific amino acid residue of Uox. To this end, the amino acid residue located at a specific position of Uox is substituted with an NNAA, and albumin may be conjugated to the substituted NNAA with a linker.

Accordingly, the NNAA according to the present invention excludes an amino acid residue playing a critical role in the activity and structure of wild-type Uox. In other words, first, an amino acid residue which does not affect the activity and structure of the wild type even when substituted with the NNAA is selected. Second, the NNAA is necessarily structurally similar to an amino acid to be substituted. Specifically, when an aromatic amino acid is substituted, an NNAA such as p-azido-L-phenylalanine (AzF), p-ethynyl-phenylalanine (pEthF), p-propargyloxyphenylalanine (pPa), O-propargyl-L-tyrosine (oPa) or L-Homopropargylglycine (HPG) is suitable. Third, the higher the relative solvent accessibility, the better. This is because the higher the solvent accessibility, the higher the possibility of conjugating albumin linked to a linker. According to a specific example of the present application, among the aromatic amino acids constituting the Uox, when two tryptophans with relatively high solvent accessibility are substituted with p-azido-L-phenylalanine (AzF), albumin may be efficiently conjugated.

The term “site-specific” used herein indicates that a carrier is specifically bound at an amino acid position to be bound to the carrier among the amino acids of Uox, and preferably, specifically bound to the amine of a tryptophan residue. When the carrier is site-specifically conjugated as described above, a problem of preparing an auxiliary conjugate with decreasing physiological activity of a long-acting formulation by connecting a carrier with an amino acid residue important for physiological activity, other than a conjugate to be prepared herein, may be prevented beforehand.

In the present application, albumin may be conjugated to the substituted NNAA using a linker, and the type of linker used may vary according to the type of substituted NNAA and the type of chemical reaction for linking NNAA with a linker. For example, when p-azido-L-phenylalanine is used as an NNAA, and a linker is linked through strain-promoted alkyne-azide cycloaddition (SPAAC), a cycloalkyne-containing linker may be used, and when p-ethynyl-phenylalanine or p-propargyloxyphenylalanine is used as an NNAA, and a linker is linked through copper catalyzed azide-alkyne cycloaddition (CuAAC), a linker having an azide group may be used. According to a specific example of the present application, when p-azido-L phenylalanine is used as an NNAA and linked through strain-promoted alkyne-azide cycloaddition (SPAAC), cyclooctyne-containing DBCO-PEG4-maleimide (DBCO-PEG4-MAL) is used as a linker. DBCO-PEG4-MAL is a heterobifunctional hydrophilic linker, and dibenzocyclooctyne (DBCO) may be chemoselectively linked to an azide functional group through strain-promoted azide-alkyne cycloaddition (SPAAC). Therefore, by genetic engineering, Uox can be manipulated to include AzF at a specific site, Uox may be allowed to react with DBCO.

SPAAC refers to strain-promoted azide-alkyne cycloaddition, and CuAAC refers to copper catalyzed azide-alkyne ring addition.

The SPAAC does not use a copper catalyst such that the functions and characteristics of a target protein are not damaged. In addition, SPAAC is activated at room temperature under an aqueous solution condition, and therefore suitable for a protein immobilization method.

In the present application, DBCO indicates dibenzocyclooctyne.

In the linkage of albumin and a linker according to the present application, the linker is bound to an amino acid residue spaced apart from an FcRn-binding domain of albumin. Since the FcRn-binding domain of albumin is a region that plays a significant role in FcRn-mediated recycling that prolongs the half-life of a protein by albumin, the linker is preferably linked to an amino acid residue spaced apart from a possible FcRn-binding domain. Preferably, compared to mono-HSA-Uox, the half-life of di-HSA-Uox is prolonged by the FcRn-mediated recycling.

In the present application, albumin and the linker may be linked by various linking methods. Preferably, the linker may be bound to cysteine 34 of the albumin, which is spaced apart from the FcRn-binding domain.

The albumin of the present application includes wild-type albumin and an albumin variant, and according to a specific example of the present application, when HSA is conjugated to Uox, an effect of significantly prolonging the serum half-life of Uox is exhibited.

The connection between the albumin and a linker may be performed prior to Step (3).

According to one embodiment of the present application, after the reaction is ended in Step (3), a desalting process using PD-10 may be further included to remove an unreacted linker. Preferably, desalting occurs at pH 5 to 8 using 10 to 30 mM sodium phosphate. More preferably, desalting occurs at pH 6 using 20 mM sodium phosphate.

According to one embodiment of the present application, the step of conjugating the Uox including an NNAA with a carrier (Step (3)) includes the following steps:

preparing HSA-PEG₄-DBCO by joining a bifunctional linker such as DBCO-PEG₄-MAL at position 34 of albumin as a carrier, wherein a molar ratio of the albumin and the linker is 1:1 to 1:8; and linking Uox including an NNAA with HSA-PEG₄-DBCO through strain-promoted azide-alkyne cycloaddition (SPAAC) to site-specifically conjugate albumin with Uox, wherein the molar ratio of the Uox and albumin is 1:1 to 1:5.

The method for manufacturing Uox including an NNAA according to the present application includes (4) separating the Uox conjugated to the carrier.

In the present application, the step of separating the Uox to which the carrier is conjugated (Step (4)) may be accomplished by 2-step, and preferably, 3-step or more chromatography. The separation of the Uox may be accomplished by sequentially performing cation exchange chromatography, anion exchange chromatography, and size exclusion chromatography.

In the present application, by sequentially performing the cation exchange chromatography, the anion exchange chromatography and the size exclusion chromatography, the most suitable enzyme that does not only increase a yield, but also produces specific Uox-albumin conjugates such as mono-HSA-Uox and di-HSA-Uox may be provided.

In the present application, a high-purity Uox-albumin conjugate may be obtained by further performing size exclusion chromatography. In addition, to separate mono-HSA-Uox in which one albumin is conjugated and di-HSA-Uox in which two albumins are conjugated, size exclusion chromatography may be used.

When the Uox separated as described above is conjugated with a carrier, the enzyme is contained at a higher purity and a higher content than Uox-HSA conjugates prepared in conventional methods, and therefore is more suitable for use as a pharmaceutic.

Specifically, the cation exchange chromatography is performed using a cation exchange resin selected from Capto-MMC, Bestarose Diamond MMC or Hitrap SP HP. Preferably, the cation exchange chromatography is performed using a Hitrap SP HP cation exchange resin. The cation exchange chromatography may be for removing unreacted albumin.

The anion exchange chromatography may be performed using an anion exchange resin selected from Capto-Adhere, Bestarose Diamond MMA or Hitrap Q HP. Preferably, the anion exchange chromatography is performed using a Hitrap Q HP resin. The anion exchange chromatography may be for removing remaining Uox.

The most widely used gels for size exclusion chromatography are series such as Sepharose (GE Healthcare), Superose (GE Healthcare), Sephadex (Pharmacia), Bio-Gel P (Bio-Rad), Superdex® (GE Healthcare) and TSKgel® (silica-based; Sigma), and according to one embodiment of the present application, Superdex 200 increase 10/300 GL may be used. The size exclusion chromatography may be to separate a material with more purity.

According to one embodiment of the present application, cation exchange chromatography and anion exchange chromatography, or size exclusion chromatography may be performed under the following conditions:

The cation exchange chromatography uses a cation exchange column, such as Hitrap SP HP. After equilibration with 20 mM sodium phosphate buffer (pH 6.0), a sample is injected, and eluted with a 0 to 100% NaCl gradient. A fraction is collected to concentrate, and then the buffer is exchanged with 20 mM Bis-Tris buffer (pH 6.5).

The anion exchange chromatography uses an anion exchange column such as Hitrap Q HP to perform elution by a NaCl gradient method. After equilibration with 20 mM Bis-Tris buffer (pH 6.5), a sample is injected, and then eluted with a 0 to 100% NaCl gradient.

For separation with more purity, size exclusion chromatography is performed. As a column, Superdex 200 increase 10/300 GL is used.

In the Step (4) of separating the Uox conjugated to the carrier according to the present invention, mono-HSA-Uox and di-HSA-Uox may be separated by size exclusion chromatography.

According to one embodiment of the present application, Step (4) of separating the Uox conjugated to the carrier includes the following steps:

performing cation exchange chromatography using a Hitrap SP HP column;

performing anion exchange chromatography using a Hitrap Q HP column; and

performing size exclusion chromatography using a Superdex 200 increase 10/300 GL column,

wherein the step of separating the Uox conjugated to the carrier may be accomplished by sequentially performing cation exchange chromatography, anion exchange chromatography and size exclusion chromatography. In addition, specifically, the step of separating the Uox conjugated to the carrier may be sequentially performing anion exchange chromatography, cation exchange chromatography and size exclusion chromatography. More preferably, in the present application, the cation exchange chromatography, anion exchange chromatography and size exclusion chromatography are sequentially performed.

The present application provides a Uox-carrier conjugate produced by the above-described method.

In the present application, the Uox produced by the above-described method includes a conjugate to which one carrier is conjugated (Mono-HSA-Uox) and a conjugate to which two carriers are conjugated (Di-HSA-Uox). They have high purity, and have a carrier efficiently conjugated to a protein to be effectively used in order to improve the half-life of a protein which is difficult to be linked to a carrier.

In addition, albumin used as a carrier is a human-derived material and has almost no immunogenicity, and therefore, immunogenicity may be reduced by conjugating Uox known to have immunogenicity with albumin.

As a carrier is conjugated with Uox, compared to only Uox, the size of the Uox-carrier conjugate increases, thereby blocking glomerular filtration and prolonging the half-life of a drug.

The Uox produced by the method according to the present application is site-specifically conjugated with a carrier, thereby maintaining drug efficacy and increasing drug persistence. Therefore, a use of the uniform conjugate has advantages in treatment and prevention of a disease.

The Uox produced by the method according to the present application may be applied to a bi-specific antibody and an antibody-drug conjugate as well as a long-acting protein therapeutic.

The Uox produced by the method according to the present application may have improved stability by conjugating two or more carriers.

Among the Uox produced by the above-describe method in the present application, a two or more carrier-conjugated conjugate may have a molecular weight ranging from approximately 230 kDa to 310 kDa. Preferably, the carrier may be a conjugate having two carriers, and have a molecular weight of approximately 250 kDa to 290 kDa. More preferably, the conjugate may be a conjugate having two carriers, and have a molecular weight of approximately 270 kDa. Still more preferably, Uox to which two HSAs are conjugated as a carrier is provided. The carriers may be Uox conjugated at sites where the above-described NNAAs are included, and preferably, at sites where tryptophan at position 160 and tryptophan at position 174 are substituted with NNAAs.

The conjugate to which two or more carriers are conjugated has a larger molecular weight and a lower glomerular filtration rate (GFR) than a conjugate to which one carrier is conjugated, thereby prolonging half-life, and when the carrier is albumin, the conjugate has two or more albumins derived from a human and having almost no immunogenicity, which is expected to reduce immunogenicity compared to a conjugate to which one albumin is conjugated.

The present application provides a pharmaceutical composition including the Uox produced by the above-described method as an active ingredient to prevent or treat one or more types of diseases selected from the group consisting of hyperuricemia, gout, deposition of urate crystals in joints, acute gouty arthritis caused by the deposition of urate crystals, urolithiasis, nephrolithiasis and gouty nephropathy. The term “hyperuricemia” used herein is a disease with a high level of uric acid in the blood. This occurs when the concentration of monosodium urate in the serum is higher than the limited solubility limit. Uric acid saturation in the plasma at 37° C. is approximately 7 mg/dl. Therefore, when this concentration is exceeded, it becomes physicochemically supersaturated. Plasma uric acid concentration is known to be relatively high when exceeding +2 S.D from the mean plasma uric acid concentration of a normal person. In most epidemiological investigations, the upper limit for males is 7 mg/dl, and the upper limit for females is 6 mg/dl. For this reason, the practical upper limit of hyperuricemia is defined as 7.0 mg/dl or more.

In the composition of the present application, the hyperuricemia and a hyperuricemia-related metabolic disorder are diseases or illnesses which occur by an increased uric acid level in the blood caused by excess uric acid remaining in the blood when an ability of the kidney to excrete uric acid is degraded since a uric acid level is higher than the normal level.

Specifically, the hyperuricemia-related metabolic disorder includes gout, uric acid crystals, deposition of urate crystals in joints, acute gouty arthritis caused by the deposition of urate crystals, monoarticular arthritis, painful seizures of inflammatory arthritis, urolithiasis, nephrolithiasis, and gouty nephropathy. Chronic nephrolithiasis and gouty nephropathy are known to increase the risk of kidney damage and renal failure.

The gout is a medical condition usually characterized by recurrent seizures of acute inflammatory arthritis, and is common in the metatarsophalangeal joint at the base of a big toe. In addition, the gout is caused by blood uric acid crystalized and deposited in joints, tendons and peripheral tissues, and may be present in the form of gout nodules, kidney stones or urate nephropathy.

The term “prevention” used herein refers to all actions of inhibiting gout, hyperuricemia, or a hyperuricemia-related metabolic disorder or delaying the onset thereof by administration of the pharmaceutical composition according to the present application.

The term “treatment” used herein refers to all actions involved in alleviating or beneficially changing symptoms of gout, hyperuricemia, or a hyperuricemia-related metabolic disorder by administration of the pharmaceutical composition according to the present application.

The composition including the Uox of the present application may contain one or more of active ingredients exhibiting the same or similar function in addition to the above-described ingredient.

The pharmaceutical composition of the present application may further include a pharmaceutically acceptable carrier in addition to the Uox as an active ingredient.

The type of carrier that can be used in the present application is not particularly limited, and any carrier that is commonly used in the art may be used. As a non-limiting example of the carrier, saline, sterile water, Ringer's solution, buffered saline, an injectable albumin solution, lactose, dextrose, sucrose, sorbitol, mannitol, xylitol, erythritol, maltitol, maltodextrin, glycerol or ethanol may be used. These carriers may be used alone or in combination of two or more thereof.

In addition, the pharmaceutical composition of the present application may be used by adding other pharmaceutically acceptable additives such as an excipient, a diluent, an antioxidant, a buffer or a bacteriostatic agent when needed, and may be used by further adding a filler, an extender, a wetting agent, a disintegrant, a dispersant, a surfactant, a binder or a lubricant.

In the pharmaceutical composition of the present application, the Uox may be included at 0.00001 wt % to 99.99 wt %, preferably, 0.1 wt % to 90 wt %, more preferably, 0.1 wt % to 70 wt %, and still more preferably, 0.1 wt % to 50 wt % with respect to the total weight of the pharmaceutical composition, but the present invention is not limited thereto. The weight percent of the Uox may vary according to the condition of an administration subject, the type of a specific symptom of disease, and the progression of a disease. When needed, the Uox may be contained at the total content of the pharmaceutical composition.

The term “administration” used herein refers to the introduction of the pharmaceutical composition of the present application to a patient by a suitable method, and the composition of the present application may be administered through various routes which can reach desired tissue, for example, orally or parenterally.

The pharmaceutical composition of the present application may be used by being formulated into various forms suitable for oral or parenteral administration.

Non-limiting examples of formulations for oral administration using the pharmaceutical composition of the present application may include troches, lozenges, tablets, an aqueous suspension, an oily suspension, formulated powder, granules, an emulsion, hard capsules, soft capsules, a syrup or an elixir.

To formulate the pharmaceutical composition of the present application for oral administration, binders such as lactose, sucrose, sorbitol, mannitol, starch, amylopectin, cellulose or gelatin; excipients such as dicalcium phosphate; disintegrants such as corn starch or sweet potato starch; or lubricants such as magnesium stearate, calcium stearate, sodium stearyl fumarate or polyethylene glycol wax may be used, and a sweetener, a fragrance or a syrup may also be used. Moreover, in the case of a capsule, a liquid carrier such as fatty oil may be further used in addition to the above-mentioned materials.

Non-limiting examples of parenteral formulations using the pharmaceutical composition of the present application may include an injectable solution, a suppository, a powder for respiratory inhalation, an aerosol for spray, an ointment, a powder for application, an oil and a cream.

To formulate the pharmaceutical composition of the present application for parenteral administration, a sterilized aqueous solution, a non-aqueous solvent, a suspension, an emulsion, a freeze-dried formulation or a formulation for external use may be used, and as the non-aqueous solvent or suspension, propylene glycol, polyethylene glycol, a vegetable oil such as olive oil, or an injectable ester such as ethyl oleate may be used.

When the pharmaceutical composition of the present application is formulated into an injectable solution, a solution or suspension may be prepared by mixing the pharmaceutical composition of the present application with a stabilizer or buffer in water, and may be formulated for unit administration of an ampoule or vial.

When the pharmaceutical composition of the present application is formulated as an aerosol, a propellant may be blended with additives to disperse a water-dispersed concentrate or wet powder.

When the pharmaceutical composition of the present application is formulated into an ointment, cream, powder for application, an oil or a dermal preparation for external use, animal oils, vegetable oils, wax, paraffin, starch, tragacanth, a cellulose derivative, a polyethylene glycol, silicone, bentonite, silica, talc or zinc oxide may be used as a carrier.

A pharmaceutically effective amount or effective dose of the pharmaceutical composition of the present application may vary according to a formulation method, administration mode, administration time and/or route of the pharmaceutical composition, and may be dependent on various factors such as the type and degree of a reaction to be achieved by the administration of the pharmaceutical composition, the type, age, body weight or general health condition of a subject to be administered, symptoms or severity of a disease, sex, diet, excretion, components of drugs or other compositions used concurrently with or at the same time or different times in the corresponding subject, and similar factors well known in the pharmaceutical field. The effective dose for desired treatment may be easily determined and prescribed by those of ordinary skill in the art. For example, the daily dose of the pharmaceutical composition of the present application may be 0.01 to 1000 mg/kg, and preferably 0.1 to 100 mg/kg, and the administration may be performed once to several times a day.

The pharmaceutical composition of the present application may be administered once or several times a day. The pharmaceutical composition of the present application may be administered separately or in combination with another therapeutic, and may be administered sequentially or simultaneously with the conventional therapeutic. Considering all of the above factors, it is important to achieve the maximum effect with the minimum dose without a side effect, and such a dose may be easily determined by one of ordinary skill in the art.

The administration route and mode of the pharmaceutical composition of the present application may be independent, and as long as the pharmaceutical composition can reach a desired corresponding site, any administration route and mode may be followed without particular limitation. The pharmaceutical composition may be administered orally or parenterally.

The parenteral administration methods of the pharmaceutical composition of the present application may include intravenous administration, intraperitoneal administration, intramuscular administration, transdermal administration or subcutaneous administration, and the composition may be applied, sprayed onto a disease site, or inhaled, but the present invention is not limited thereto.

The pharmaceutical composition of the present application may also be used in combination with various methods such as hormone treatment and drug treatment to prevent or treat gout, hyperuricemia, or a hyperuricemia-related metabolic disorder.

The present application provides a food composition for preventing or improving one or more diseases selected from the group consisting of hyperuricemia, gout, deposition of urate crystals in joints, acute gouty arthritis caused by the deposition of urate crystals, urolithiasis, nephrolithiasis and gouty nephropathy, which includes the Uox produced by the above-described method as an active ingredient.

The term “improvement” used herein refers to all actions involved in alleviating or beneficially changing a disease by administration of the pharmaceutical composition according to the present application.

The food composition of the present application may be used as a health functional food. The “health functional food” refers to a food which is manufactured and processed using a raw material or ingredient having functionality useful to a human body according to the Health Functional Food Act No. 6727, and the “functionality” refers to ingestion or intake for the purpose of controlling nutrients or obtaining a useful effect for health such as a physiological action in terms of the structure or function of the human body.

The food composition of the present application may include an additional component that is generally used to improve odor, taste or appearance. For example, the food composition may include vitamins A, C, D, E, B1, B2, B6 and B12, niacin, biotin, folate, and pantothenic acid. In addition, the food composition may include a mineral such as zinc (Zn), iron (Fe), calcium (Ca), chromium (Cr), magnesium (Mg), manganese (Mn) or copper (Cu). The food composition may further include an amino acid such as lysine, tryptophan, cysteine or valine. The food composition may further include food additives such as a preservative (calcium sorbate, sodium benzoate, salicylic acid or sodium dihydroacetate), a disinfectant (bleaching powder or highest bleaching powder or sodium hypochlorite), an antioxidant (butyl hydroxyanisole (BHA) or butylhydroxytoluene (BHT)), a dye (tar dye), a coloring agent (sodium nitrite), bleach (sodium sulfite), a seasoning (sodium glutamate (MSG)), a sweetening agent (dulcin, cyclemate sodium, saccharin sodium), a flavor (vanillin or lactones), a leavening agent (alum or potassium D bitartrate), a reinforcing agent, an emulsifying agent, a thickening agent, a coating agent, a gum base, a foam inhibitor, a solvent, and an improving agent. The additives may be selected according to food type and used in a suitable amount.

When the food composition of the present application is used as a food additive, it may be added as it is or used in combination with another food or food component, and may be appropriately used according to a conventional method.

In the food composition of the present application, the Uox content is not particularly limited, and may vary according to the state of an administration subject, the type of specific symptom or degree of progression. When needed, the food composition may be included at the total content of a food.

### Advantageous Effects

A method of preparing Uox including a non-natural amino acid of the present application can be effectively used to prolong the half-life of a protein which is difficult to be linked to a carrier, by efficiently linking the carrier to the protein.

In addition, the Uox produced by the above-described method can maintain the efficacy of a drug due to a carrier selectively conjugated to a specific site, increase drug persistence, reduce the risk of an immune response, and facilitate separation due to the generation of a uniform conjugate, and thus can be effectively used in various biopharmaceuticals.

### DESCRIPTION OF DRAWINGS

FIG. 1 is a diagram showing a result of comparing conventional Uox-W160, 174AzF (6xHis)(template) and a His-tag-removed sequence (deletion) of an Uox expression vector.

FIG. 2 shows a fed-batch culture profile of an Uox-producing strain.

FIG. 3 shows a hydrophobic chromatography result for Uox separation.

FIG. 4 shows an anion exchange chromatography for Uox separation.

FIGS. 5 and 6 show a size exclusion chromatography result for Uox separation, an SDS-PAGE gel image, and an SEC-HPLC result. Specifically, FIG. 5 shows a size exclusion chromatography for Uox separation and an SDS-PAGE gel image.

FIG. 6 shows a size exclusion chromatography-high-performance liquid chromatography (SEC-HPLC) result for analyzing Uox purity.

FIG. 7 shows a result of confirming AzF introduction of Uox through SDS-PAGE analysis.

FIG. 8 shows a cation exchange chromatography result for separation of an Uox-carrier conjugate.

FIG. 9 shows an anion exchange chromatography result for separation of an Uox-carrier conjugate.

FIGS. 10 and 11 shows a size exclusion chromatography result and an SDS-PAGE gel image for separating an Uox-carrier conjugate. Specifically, FIG. 10 shows size exclusion chromatography results of mono-HSA-Uox and di-HSA-Uox according to a reaction molar ratio of Uox:HSA-DBCO=1:1. FIG. 11 shows size exclusion chromatography results of mono-HSA-Uox and di-HSA-Uox according to a reaction molar ratio of Uox:HSA-DBCO=1:3.

FIG. 12 shows a result of separating mono-HSA-Uox and di-HSA-Uox, analyzed through SEC-HPLC.

FIG. 13 shows an effect of reducing a blood uric acid level of an Uox-carrier conjugate in a hyperuricemia mouse animal model.

FIG. 14 shows an effect of reducing a blood uric acid level of a Uox-carrier conjugate in a hyperuricemia rat animal model.

FIGS. 15 to 17 show kidney autopsy results of a hyperuricemia rat animal model. Specifically, FIG. 15 shows the appearances of the kidneys of animal models.

FIGS. 16 and 17 show histomorphological changes in animal models.

FIG. 18 shows an anion exchange chromatography result for Uox separation in a second Uox separation and purification process.

FIG. 19 shows a hydrophobic interaction chromatography result for Uox separation in a second Uox separation and purification process.

FIGS. 20 and 21 show a size exclusion chromatography result, an SDS-PAGE gel image and a SEC-HPLC result for Uox separation in a second Uox separation and purification process. Specifically, FIG. 20 shows the size exclusion chromatography result and SDS-PAGE gel image for Uox separation. FIG. 21 shows the SEC-HPLC result for analyzing Uox purity.

FIG. 22 shows the result of measuring in vitro enzyme activity of Uox-HSA.

FIG. 23 shows the comparison of the effect of reducing a blood uric acid level between Uox-HSA and competing drugs in hyperuricemia rat animal models.

FIG. 24 shows the comparison of the pharmacodynamic effect between Uox-HSA and competing drugs in animal models.

FIG. 25 shows an immunogenic analysis result in animal models.

FIGS. 26 and 27 show the molecular weight analysis results of Di-HSA-Uox.

FIGS. 28 and 29 show the results of analyzing the isoelectric point (pI) characteristic of Uox-HSA using cIEF.

FIG. 30 shows the comparison of a pharmacodynamic effect between mono-HSA-Uox and di-HSA-Uox in SD-Rats.

### MODES OF THE INVENTION

1. Urate Oxidase (Uox)

A Urate oxidase (hereinafter, Uox) is an enzyme having a function of degrading uric acid. Since the human body does not produce the Uox, if uric acid is not smoothly degraded, gout may occur. The Uox may be used as a material for treating a disease caused by uric acid, representatively, gout. The Uox is a tetramer having a form in which four monomers with the same structure are linked.

In one embodiment, Uox may be Aspergillus Flavus-derived Uox. Here, the structure of an Aspergillus Flavus-derived Uox monomer present in nature is SEQ ID NO: 1. In another embodiment, Uox may be microorganism-derived Uox. In still another embodiment, Uox may be mammal-derived Uox.

In the present application, urate oxidase is indicated as an Uox.

The Uox has a form in which four Uox monomers (p′) are linked together. In “p” according to the present application, one or more residues of the amino acid sequence of SEQ ID NO: 1 are substituted with NNAAs.

In one embodiment, the “p′” may have a modified structure in which one or more amino acid residues selected from the group consisting of tyrosine at position 8, tyrosine at position 16, tyrosine at position 30, tyrosine at position 46, tyrosine at position 65, phenylalanine at position 79, phenylalanine at position 87, tyrosine at position 91, tryptophan at position 106, phenylalanine at position 120, phenylalanine at position 159, tryptophan at position 160, phenylalanine at position 162, tyrosine at position 167, tryptophan at position 174, tryptophan at position 186, tryptophan at position 188, phenylalanine at position 191, phenylalanine at position 204, tryptophan at position 208, phenylalanine at position 219, tyrosine at position 233, tyrosine at position 251, tyrosine at position 258, phenylalanine at position 259, tryptophan at position 265 and phenylalanine at position 279 of the amino acid sequence of SEQ ID NO: 1 are substituted with NNAAs. Further, the “p′” may have a structure in which tryptophan at position 160 or phenylalanine at position 174 of the amino acid sequence of SEQ ID NO: 1 are substituted with an NNAA.

In one embodiment, the substituted NNAA may be p-azido-L-phenylalanine (AzF), p-ethynyl-phenylalanine (pEthF), L-homopropargylglycine (HPG), 0-propargyl-L-tyrosine (oPa) or p-propargyloxyphenylalanine (pPa). Further, the substituted NNAA may be p-azido-L-phenylalanine (AzF).

2. Crosslinker

The scope of the present application includes a crosslinker for conjugating Uox with albumin. The term “crosslinker” used herein is an agent for connecting two proteins, as well as a structure of connecting two proteins in the structure of a conjugate formed by a reaction.

In the structural formula of the present application, the crosslinker is indicated as L.

The crosslinker as an agent consists of a functional group for forming a bond with Uox, a functional group for forming a bond with albumin and a structure of connecting them. After forming a conjugate, the functional groups correspond to a connecting structure between Uox and the crosslinker and a connecting structure between the crosslinker and albumin of the conjugate structure.

In one embodiment, the crosslinker may include a functional group reactive to Uox to be connected with Uox. Further, the functional group reactive to Uox may be a click-chemistry functional group. Furthermore, the click-chemistry functional group may be dibenzocyclooctyne (DBCO), azide, tetrazine, or transcyclooctene. Furthermore, the click-chemistry functional group may be dibenzocyclooctyne.

In one embodiment, the crosslinker may include a functional group reactive to albumin to be connected with albumin. In one example, the reactive functional group may have reactivity to a thiol (—SH) of a cysteine residue. Here, the reactive functional group includes maleimide (MAL), 3-arylpropiolonitrile, haloacetal, pyridyl disulfide, and conventionally used ones. In another example, the reactive functional group may have reactivity to (—NH₂) of a lysine residue. Here, the reactive functional group includes N-hydroxysuccinimide ester (NHS), imidoester, and conventionally used ones. In still another example, the reactive functional group may be a click-chemistry functional group.

A structure in which two functional groups of a crosslinker or a structure of connecting structures including the same will be described. In one embodiment, the structure may include an alkylene, alkenylene, alkynylene, aralkylene, arylalkylene or (CH₂O)ₙ. The terms “alkylene,” “alkenylene,” “alkynylene,” “aralkylene,” and “arylalkylene” refer to conventionally understood structures including a hydrocarbon chain and/or an aromatic ring, and are intended to also include all structures including allowable substitutions or hetero atoms. Further, the structure may include (CH₂O)ₙ. Furthermore, the structure may include (CH₂O)₁₀.

3. Carrier

A carrier connected to the conjugate of the present application refers to a material such as a protein or polymer with a longer half-life in the body. In one embodiment, the carrier may be a serum protein. The serum protein is keyhole limpet hemocyanin (KLH), globulin or albumin, which is found in serum and has a longer half-life. In one embodiment, the carrier may be a polymer. In one example, the carrier may be polyethylene glycol (PEG).

In one embodiment, the carrier may be albumin. Here, the albumin refers to commonly referred to albumin. In one embodiment, the albumin may be mammal-derived albumin. Further, albumin may be human serum albumin (HSA). In one embodiment, the albumin may be wild-type. In another embodiment, the albumin may be genetically manipulated. Further, the genetic manipulation may be manipulation to include NNAA. The “wild type” or “genetically manipulated” used herein is a term indicating a protein state determined on the basis of whether a naturally-found protein and an amino acid sequence are the same.

In the structural formula of the present application, the carrier is represented as Car, and the albumin is represented as Alb.

4. Uox-Carrier Conjugate

An Uox-carrier conjugate having the structure of following structural formula 1 according to the present invention is provided:

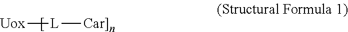

In Structural Formula 1, the description of components Uox, L and Car may be applied mutatis mutandis to the above description of 1., 2. and 3.

Here, n may be 1 to 8. Further, n may be 1 to 4. Preferably, n is 1 or 2. Furthermore, n may be 1. In another example, n may be 2.

In one example, when n=1, Uox may consist of three p′ monomers to which a carrier is not connected and one p′ monomer to which one carrier is connected. In one example, when n=2, Uox may consist of three p′ monomers to which a carrier is not connected and one p′ monomer to which two carriers are connected. In another example, when n=2, Uox may consist of two p′ monomers to which a carrier is not connected and two p′ monomers to which a carrier is connected.

In one embodiment, Car may be albumin. Here, a Uox-albumin conjugate having the following Structural Formula 2 is provided:

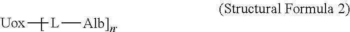

The description of components Uox, L and Alb may be applied mutatis mutandis to the above description of 1., 2. and 3., and also applied to the description of Structural Formula 1.

4.1. Click-Chemistry

The term “click-chemistry” is a chemical concept introduced by K. Barry Sharpless of the Scripps Research Institute to explain a complementary chemical functional group and a chemical reaction, which are designed for two molecules to rapidly and stably make a covalent bond. The click-chemistry refers to a specific reaction, as well as the concept of a rapid and stable reaction. In certain embodiments, the click-chemistry is modularized, has a wide range and high yield, does not have a significant by-product, is stereospecific and physiologically stable, has a great thermodynamic driving force (e.g., >84 kJ/mol), and/or has to have high atom economy. Several reactions are known to satisfy the above conditions:

(1) Huisgen 1,3-dipolar cycloaddition (e.g., including a Cu(I)-catalytic cycloaddition reaction, and also frequently referred to generically as a “click reaction”; see Tornoe et al., Journal of Organic Chemistry (2002) 67: 3057-3064): copper or ruthenium is generally used as a catalyst;

[Schematic Diagram of Huisgen 1,3-Dipolar Cycloaddition]

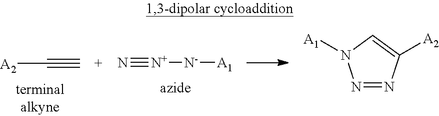

(2) Diels-Alder reaction, which is a cycloaddition reaction including, for example, a normal electron-demand Diels-Alder reaction and an inverse electron-demand Diels-Alder reaction, but not limited thereto (i.e., strain-promoted cycloaddition (SPAAC));

[Schematic Diagram of Diels-Alder Reaction]

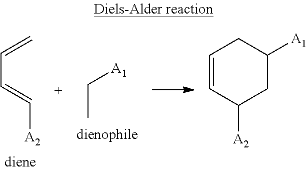

[Example of Diels-Alder Reaction; TCO and Tetrazine]

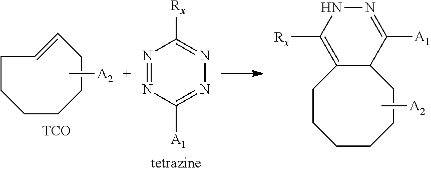

[Schematic Diagram of Strain-Promoted Cycloaddition Reaction]

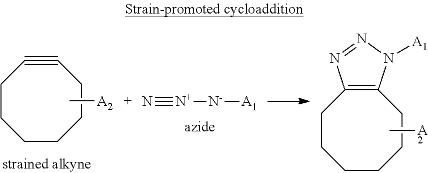

[Example of Strain-Promoted Cycloaddition Reaction; Azide and DBCO]

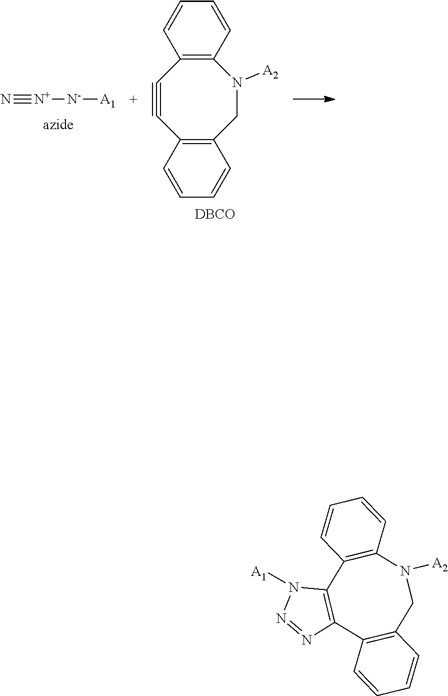

(3) Nucleophilic addition for small stained rings such as epoxide and aziridine;

(4) Nucleophilic addition for activated carbonyl group;

(5) Addition reaction for carbon-carbon double bond or triple bond.

[Addition Reaction of Thiol and Alkene]

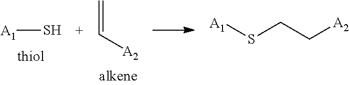

The term “click-chemistry functional group” used herein refers to a functional group involved in the click-chemistry. For example, a strained alkyne, for example, cyclooctyne, corresponds to a click-chemistry functional group. Generally, the click-chemistry needs at least two molecules including complementary click-chemistry functional groups, respectively. A pair of click-chemistry functional groups having reactivity is referred to as a “partner click-chemistry functional group” in the present application. For example, in the strain-promoted cycloaddition reaction between azide and cyclooctyne, the azide is a partner click-chemistry functional group of cyclooctyne and other alkynes. Exemplary click-chemistry functional groups used in the present application include a terminal alkyne, an azide, a strained alkyne, a diene, a dienophile, a trans-cyclooctene, an alkene, a thiol and tetrazine, but the present invention is not limited thereto. Other click-chemistry functional groups are known to those of ordinary skill in the art.

4.2. Linkage of Uox and Crosslinker

In Structural Formula 1, the connecting relationship between Uox and L will be described in further detail. The Uox and L are linked together by a functional group reactive to Uox included in L.

In one embodiment, the Uox and the crosslinker may be linked by a modified NNAA of the p′ as described in 1.

In one embodiment, the connecting structure of Uox and the crosslinker may be a binding structure formed by click-chemistry. Since an NNAA including a click-chemistry group can be inserted into Uox according to the preparation process of the present application, a conjugate can be prepared using click-chemistry. Since the click-chemistry and its structure is bio-orthogonally formed, the reaction is stably performed and the structure is not easily broken. In one example, the connecting structure of Uox and the crosslinker may be a binding structure formed by a reaction between dibenzocyclooctyne (DBCO) and azide (see 4.1.). Since the NNAA AzF includes azide, as an example, it can be used to form the binding structure.

4.3. Linkage of Crosslinker and Albumin

In Structural Formula 1, the connecting relationship between L and Alb will be explained in further detail. L and Alb are linked together by a functional group of L, reactive to albumin.

In one embodiment, the crosslinker and the albumin may be connected by a cysteine residue of the albumin. Since the cysteine residue includes a thiol (—SH) having reactivity, it is generally used to prepare a bioconjugate. Here, the connecting structure of the crosslinker and the albumin may be a binding structure formed by the reaction between the functional group and the cysteine residue of the albumin as described in 2. In one example, the connecting structure may be a binding structure formed by a reaction between maleimide (MAL) and a cysteine residue of the albumin.

In one embodiment, the crosslinker and the albumin may be connected by a lysine residue of the albumin. Since the lysine residue includes a free amine group (—NH₂) having reactivity, it is generally utilized to prepare a bioconjugate. Here, the connecting structure between the crosslinker and the albumin may be a binding structure formed by a reaction between the functional group and a cysteine residue of the albumin as described in 2.

However, since the present application is characterized by a binding site that does not disturb the activity of Uox and a click-chemistry reaction used for the corresponding binding, the binding between the albumin and the crosslinker corresponds to a part with a low need for problem solving. Therefore, the connecting structure may use any connecting method that can be generally used. The present application is described with the intention not to be greatly limited by the binding structure between the crosslinker and the albumin.

5. Efficacy of Uox-Albumin Conjugate

This section is intended to explain the improved efficacy when a Uox-albumin conjugate having the structure that is able to be characterized by the above-described 1. and 4. is compared with wild-type Uox. Further, this section is for explaining the improved efficacy when being compared with other persistent Uox drugs.

5.1. Maintenance and Improvement of Urea Degrading Activity

Generally, a persistent drug prepared by connecting a macromolecule and a protein is greatly decreased in activity. This is because there is a problem in which the connected macromolecule blocks the active site of a target protein. This problem can be analyzed in two aspects. (1) In the case of a technique of connecting a macromolecule through random substitution such as PEGylation in the conventional art, a connection site of the macromolecule is not specified. For this reason, it is impossible to connect a macromolecule by avoiding the active site. (2) Although specific binding is possible, much research is required to find a connection site which does not suppress the activity of a desired protein. Substitution should occur at a site away from the active site of the desired protein, at the time, a site at which the formation of the tertiary structure of the protein is not inhibited has to be substituted, as well as a site with high solvent accessibility for a high yield. At the same time, since the mode of action of a desired protein in the human body may not be completely defined, it is necessary to find a binding site that does not inhibit additional interactions such as various coenzymes. To this end, efforts are required to select candidate substitution sites according to certain criteria, and to actually experiment with them to select sites with high activity.

As confirmed in the following Examples 6, 11 and 12, the Uox-albumin conjugate according to the present application (hereinafter, Uox-HSA) has the same or improved activity compared with wild-type Uox. As shown in Table 6, Uox-HSA has similar activity to a wild-type protein (Fasturtec) compared at the same dose, and exhibits activity two-fold or higher than a persistent drug such as KRYSTEXXA. In consideration that the conventional persistent drug does not have as much efficacy compared with the wild-type protein, this effect is very significant. As described above, by blocking an active site, the activity of KRYSTEXXA prepared through random PEGylation was greatly lowered. In addition, having similar activity to the wild-type protein means that the tertiary structure of the protein is completely maintained without blocking the active site, and other additional actions, in addition to a direct reaction with a substrate, are not inhibited.

5.2. Enhancement of Persistency

Since the Uox-HSA of the present application includes albumin participating in an FcRn cycle, a half-life is greatly improved compared with a wild-type protein. As shown in Example 13, Uox-HSA has a half-life 8.7-fold or longer than the wild-type protein. In addition, the effect of enhancing the persistency of Uox-HSA shows a similar value compared with a conventional drug, such as KRYSTEXXA. Therefore, it can be confirmed that the Uox-HSA of the present application has greatly increased persistence, as intended.

Since the molecular weight of KRYSTEXXA is much greater than Uox-HSA (KRYSTEXXA: 497 kDa, Uox-HSA: 270 kDa), and Uox-HSA uses HSA and thus is expected to further prolong a half-life when being used for a human, efficacy may be considered more positively.

5.3. Decrease in Immunogenicity

Uric acid oxidases mainly used for medical purposes are microorganism-derived proteins. Due to the characteristics of a foreign protein, these uric acid oxidases have high immunogenicity. Therefore, when they are administered to the human body, there is a problem in that they can be degraded by an immune response or symptoms of the immune response may occur. In addition, there is also a problem in that a dosage has to be adjusted to avoid these immune diseases.

Albumin accounts for the majority of serum proteins. For this reason, albumin is a protein which is very stable and exhibits almost no immunogenicity. As a foreign protein Uox is conjugated with albumin, an effect of reducing the immunogenicity of Uox may be expected.

According to Example 15, it was possible to confirm that Uox-HSA is actually decreased in immunogenicity compared to a wild-type protein. Further, in consideration that Uox-HSA uses HSA, when being applied to the human body, it can be expected to show a more improved effect.

5.4. Di-HSA-Uox has Improved Efficacy Compared with Mono-HSA-Uox

Di-HSA-Uox of Uox-HSAs according to the present application has improved efficacy compared with mono-HSA-Uox. Since the present application has a half-life prolonging effect and an immunogenicity improving effect by the albumin conjugation, it was expected that as the number of albumins increases, the half-life and immunogenicity would be further improved. In addition, considering preparation difficulty and steric hindrance, it was thought that it would be most appropriate to conjugate two albumins. As a result of verifying this, as confirmed in Examples 14 and 15, it was confirmed that a conjugate to which two albumins are conjugated has more improved physical properties. The di-HSA-Uox according to the present application is a novel material which has not been previously revealed, and has excellent efficacy as described above.

Hereinafter, the present application will be described in further detail with reference to examples. However, these examples are provided to exemplify the present application, and the scope of the present application is not limited to these examples.

Hereinafter, experimental materials used in the present application are as follows: p-azido-L-phenylalanine (AzF) was purchased from Chem-Impex International (Wood Dale, Ill.), human serum-derived albumin was purchased from Albumedix (Nottingham, UK), a Vivaspin centrifuge concentrator was purchased from Sartorius Corporation (Bohemia, N.Y.), DBCO-PEG₃-FITC was purchased from Conju-Probe, LLC (San Diego, Calif.), DBCO-PEG₄-MAL was purchased from Click-Chemistry Tools (Scottsdale, Ariz.), and a PD-10 desalting column, a Hitrap phenyl FF hydrophobic interaction column, a HiTrap Q HP cation exchange column, a HiTrap SP HP anion exchange column, a Superdex 200 10/300 GL size exclusion column and AKTA pure 25 L were purchased from GE Health Care (Piscataway, N.J.). As all other chemical reagents, products from Sigma-Aldrich Corporation (St. Louis, Mo., USA) were used.

### Example 1. Construction of Expression Vector from which Six his were Removed for Producing Urate Oxidase

An expression cell line (developed in Prior Patent No. KR 10-1637010) for constructing conventional Uox is inappropriate as a commercial strain because of immunogenicity and the possibility of having an unpredictable side effect in future non-clinical or clinical trials due to six histidines (His) attached to the C-terminus. Accordingly, a commercial strain from which six His are removed was constructed.

As a vector into which a non-natural amino acid can be inserted, a pEVOL-pAzF plasmid (Plasmid ID: 31186) including an AzF-specific engineered pair consisting of a tyrosyl-tRNA synthetase originating from Methanococcus jannaschii and amber suppressor tRNA was purchased from Addgene (Cambridge, Mass.) and used without additional modification.

To construct an expression vector from which six His are removed from the C-terminus of the conventional pQE80-Uox plasmid, the following primers were used:

F:
(SEQ ID NO: 6)
5-CTCTCTGAAGAGCAAGCTGTAAGCTTAATTAGCTGAGC-3
R:
(SEQ ID NO: 7)
5-GCTCAGCTAATTAAGCTTACAGCTTGCTCTTCAGAGAG-3

To substitute tryptophansat positions 160 and 174 of Uox with an amber codon (UAG), site-directed mutagenic PCR was performed using the pQE80-Uox as a template. To introduce the amber codon at position 160, 5-CAATTCACAGTTTTAGGGGTTTCTGAG-3 and 5-CTCAGAAACCCCTAAAACTGTGAATTG-3 primers were used, and to introduce the amber codon at position 174, 5-CACTGAAGGAGACTTAGGATAGAATCCTG-3 and 5-CAGGATTCTATCCTAAGTCTCCTTCAGTG-3 primers were used.

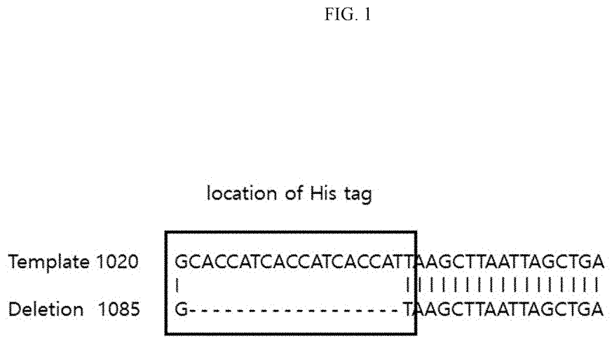

A result of sequencing for the used strain is shown in FIG. 1.

As confirmed from FIG. 1, it was confirmed that a His-tag was removed from the strain.

### Example 2. Fed-Batch Culture Process of UoX Producing Strain

A fed-batch process that provides a carbon source and a nitrogen source for mass production of a non-natural amino acid-introduced Uox was performed. The strain was E. Coli C321.ΔA.exp[(pEVOL-AzF)(pQE80-Uox.W160,174amb)]. The E. Coli C321.ΔA.exp[(pEVOL-AzF)(pQE80-Uox.W160,174amb)] was prepared by simultaneously transforming E. coli C321.ΔA.exp(Addgene, ID: 49018) with pEVOL-pAzF and the pQE80-UoxW160.174amb of Example 1, and is hereinafter referred to as E. Coli C321.ΔA.exp[(pEVOL-AzO(PQE80-Uox.W160,174amb)].

The bioreactor was a 75 L bioreactor (Sartorius Corporation, Bohemia, N.Y.). Seed culture was accomplished twice, and started with adjustment of a medium volume of the main culture to 30 L. The culture was performed under conditions of 30° C., pH 7.0, 600 rpm, 1 vvm and 100% DO, and during culture, oxygen was continuously provided. For primary seed culture, a medium (12 g/L tryptone, 24 g/L yeast extract, 5 g/L glycerol, 2.3 g/L KH₂PO₄, 12.53 g/L K₂HPO₄, 100 μg/mL ampicillin, 35 μg/mL chloramphenicol) was prepared and inoculated (0.75%), and culture was carried out at 37° C. and 200 rpm for 15 hours. For secondary seed culture, 5% of the primary seed culture liquid was inoculated, and then culture was carried out at 30° C., 500 rpm, 1 vvm and 100% DO to an O.D. of 5 or more. For the main culture, a medium (12 g/L tryptone, 12 g/L yeast extract, 3.2 g/L KH₂PO₄, 17.4 g/L K₂HPO₄, 100 μg/mL ampicillin, 35 μg/mL chloramphenicol and 0.1 g/L thiamine) was prepared and inoculated with 13.3% of the secondary seed culture liquid, and culture was carried out at 30° C., pH 7.0, 600 rpm, 1 vvm and DO 100%, and when O.D. reached 140 or more, 2 mM AzF, 1 mM IPTG and 0.2% arabinose were added to induce protein expression. In addition, cell growth was induced while an additional carbon source medium (600 g/L glucose, 1.2 g/L MgSO₄) and an additional nitrogen source medium (240 g/L yeast extract, 1.5 g/L ammonium sulfate) were provided at a constant rate at the time point of the concentration of a carbon source decreasing. After the culture, a pellet was obtained by centrifugation at 4° C. and 6,000 rpm for 10 minutes, and stored at −80° C. for a subsequent experiment.

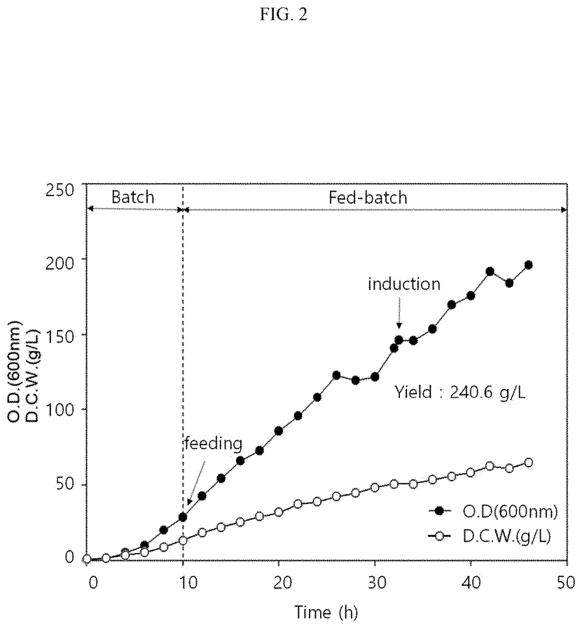

The result is shown in FIG. 2.

As confirmed in FIG. 2, when E. coli cells proliferate to a certain level, growth inhibition occurs due to an increasing pH or depletion of a carbon source, which is a nutrient in a medium, and if it is left unattended, cell disruption is caused. To solve this, the cells were cultured using fed-batch culture providing an additional medium at a constant rate. An initial main culture medium was adjusted to 30 L for culture, and when O.D. reached 140 or more, for AzF and overexpression induction, IPTG and arabinose were added. After ten hours of culture, which is the time when almost all of a carbon source, glucose, is consumed, fed-batch culture was carried out while the additional medium was provided at a constant rate. As a result, after 46 hours, O.D. reached 195.9, and the final yield was 240.6 g/L.

### Example 3. Separation and Purification Process for AzF-Introduced Uox-1

The present application introduces two types of processes for separating and purifying AzF-introduced Uox. A first process is described in detail in Example 3, and the other process is described in detail in Example 8.

To separate and purify Uox from the collected pellet, three-step chromatography was performed. The pellet was suspended in a suspension buffer (20 mM Tris-HCl pH 8.5, 1 M (NH₄)₂SO₄, and 1 mg/mL of lysozyme was added to be dissolved for 30 minutes. A supernatant was obtained by centrifugation at 10,000 rpm for 20 minutes, followed by purification through chromatography. In the first step, a 1 M (NH₄)₂SO₄-containing solvent was equilibrated using a hydrophobic column (Hitrap phenyl FF) by hydrophobic interaction chromatography, a sample was injected thereinto, followed by elution with a (NH₄)₂SO₄-free solvent. After concentration, the buffer was exchanged with 20 mM Tris-HCl pH 8.5, and then two-step anion exchange chromatography was performed. Elution was performed by an NaCl gradient method using an anion change column (Hitrap Q HP). Subsequently, to increase purity, three-step size exclusion chromatography was performed, separation was performed using a size exclusion column (Superdex 200 10/300 GL), and then analysis was performed using SDS-PAGE and SEC-HPLC columns (Shodex LW803, 8.0×300 mm, 3 μm) to confirm purity. For a comparative experiment, culture and separation were performed by the same method as described above, except an AzF injection method using E. Coli Top10 (pQE80-Uox-WT), to produce Uox-WT.

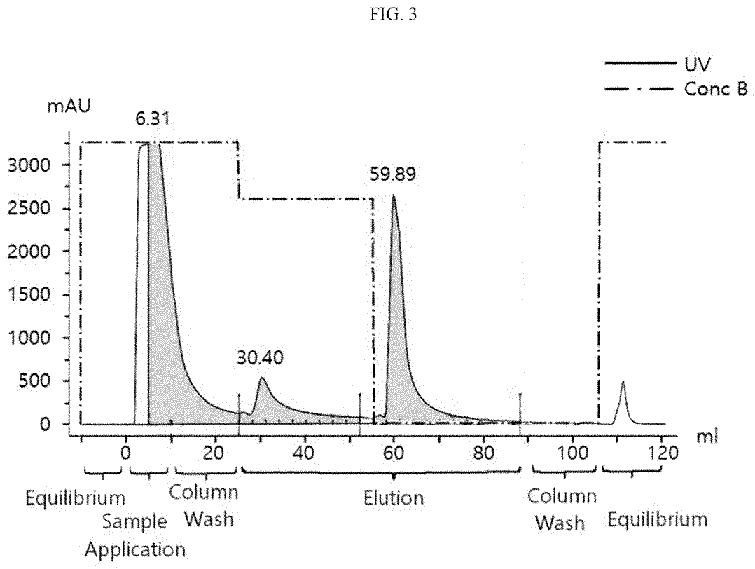

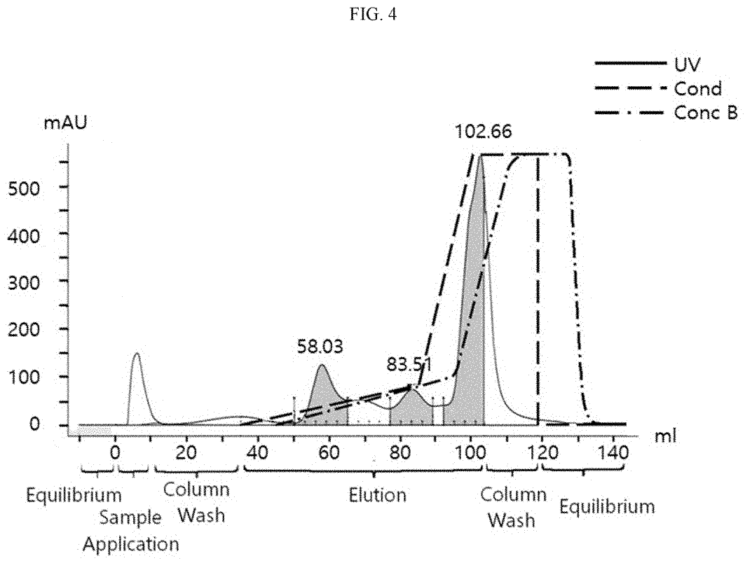

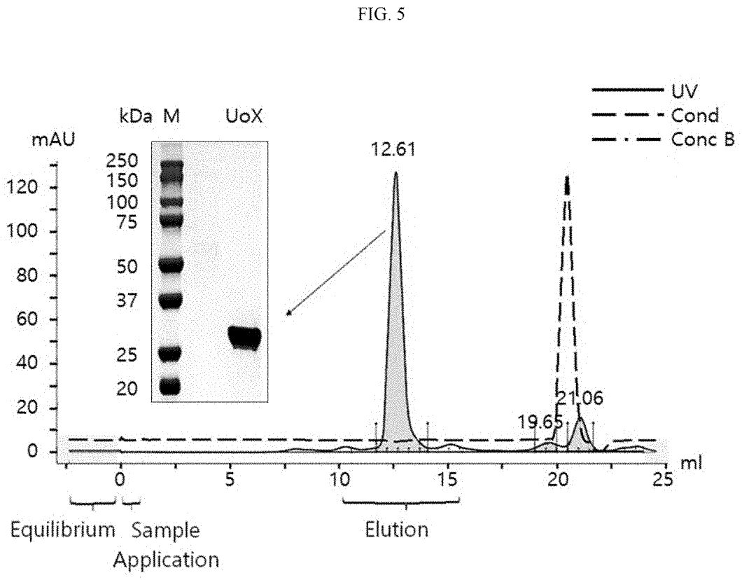

The results are shown in FIGS. 3 to 6.

As confirmed in FIGS. 3 to 5, the total yield of Uox was 1 to 10 mg/g.

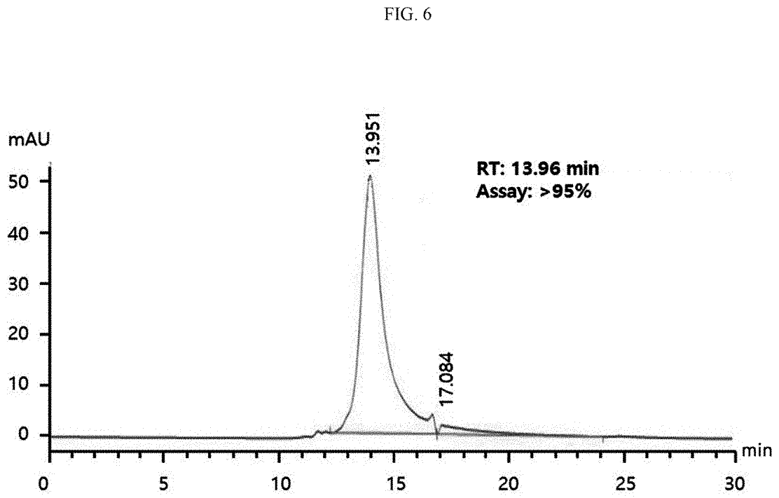

As confirmed in FIG. 6, as analyzed by SEC-HPLC, Uox with 95% or more purity was separated.

To detect an endotoxin and impurities in the Uox separated and purified with high purity, the presence of a bacterial toxin was determined by an Endotoxin Kinetics method, and for host-derived protein analysis, the impurities were confirmed and quantified using a host cell protein kit.

As a result, the endotoxin was detected at 5 EU/mg or less, and HCP was detected at 10 ppm or less in the Uox separated and purified with high purity.

### Example 4. Confirmation of Whether AzF of UoX was Introduced Using Strain-Promoted Alkyne-Azide Cycloaddition (SPAAC

Each of Uox-WT and Uox-AzF, each of which was adjusted to a concentration of 10 μM, and 80 μM DBCO-PEG₃-FITC were reacted for 2 hours, and then unreacted DBCO-PEG₃-FITC was removed using a PD-10 column, followed by loading on an SDS-PAGE gel. The reaction with DBCO-PEG₃-FITC was analyzed through SDS-PAGE, and visualized using a Blue/white transilluminator (Bioneer, Daejoen). A triazole was formed by bonding of an azide group and DBCO, and AzF introduction to Uox was verified using the principle that fluorescein isothiocyanate (FITC) is excited in a blue light (470 nm) range to exhibit green.

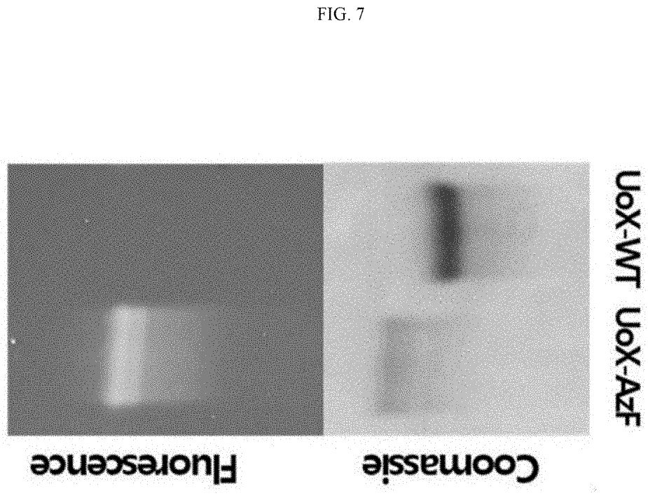

The result is shown in FIG. 7.

As confirmed in FIG. 7, Uox-WT did not exhibit fluorescence, and Uox-AzF exhibited strong fluorescence. This indicates that AzF is introduced at specific positions (W160 and W174) of Uox.

### Example 5. Preparation and Separation Processes of Conjugate of HSA and Uox

As a carrier, human serum albumin (HSA) was used, and only a buffer (PBS pH 7.4) was exchanged without a separate separation process. Cysteine at position 34 of HSA was connected with a bifunctional linker DBCO-PEG₄-MAL through Michael addition, thereby preparing HSA-PEG₄-DBCO. A reaction molar ratio of the albumin and the bifunctional linker DBCO-PEG₄-MAL was 1:1, 1:2, 1:4 or 1:8, and these materials were reacted at room temperature for 2 hours. After the reaction, for removal of a remaining linker and exchange of the buffer, the buffer was exchanged with 20 mM sodium phosphate pH 6.0 using a PD-10 column.

As a result of confirming the conjugation yield of HSA-PEG₄-DBCO, in a 1:4 reaction, a yield of 93.2% was able to be obtained, and in a 1:8 reaction, a yield of 103.9% was able to be obtained. Considering the high price of the bifunctional linker DBCO-PEG₄-MAL, a 1:4 molar ratio of the albumin and the bifunctional linker was used for an experiment.

To connect HSA site-specifically to Uox-AzF, a conjugate of Uox and HSA was prepared using strain-promoted azide-alkyne cycloaddition (SPAAC). A click-chemistry reaction for forming a stable triazole by bonding an azide group and DBCO under a Cu-free condition was used. The azide group-introduced Uox-AzF was reacted with HSA-PEG₄-DBCO in a molar ratio of 1:1, 1:1.5, 1:2 or 1:3 at room temperature and then a conjugation yield was calculated by three-step chromatography. In the first step, to remove unreacted albumin, a cation exchange column (Hitrap SP HP) was used. After equilibration with 20 mM sodium phosphate pH 6.0, a sample was injected, and eluted with a 0 to 100% NaCl gradient. Fractions were collected and concentrated, and then the buffer was replaced with 20 mM Bis-Tris pH 6.5. In the second step, to remove remaining Uox, an anion exchange column (Hitrap Q HP) was used. After equilibration with 20 mM Bis-Tris pH 6.5, a sample was injected, and then eluted with a 0 to 100% NaCl gradient. Fractions were collected and concentrated, and then in the third step, mono-HSA-Uox and di-HSA-Uox were purely separated using a size exclusion column (Superdex 200 10/300 GL), and purity was confirmed by SEC-HPLC and SDS-PAGE.

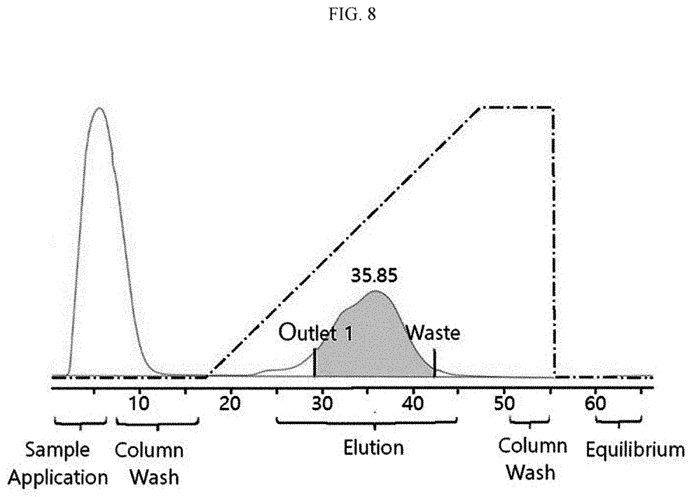

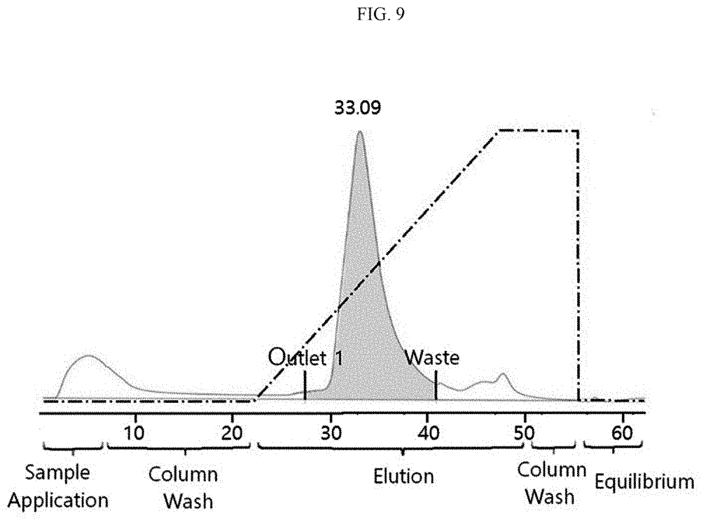

The results are shown in Table 1 and FIGS. 8 to 12.

TABLE 1
Conjugation yield according to molar
ratio of HSA-DBCO and Uox

| HSA-DBCO:Uox | Conjugation |
|---|---|
| (molar ratio) | yield (%) |
| 1:1 | 5.3 |
| 1.5:1 | 9.2 |
| 2:1 | 11.0 |
| 3:1 | 9.3 |

As confirmed in Table 1, the conjugation yield according to the molar ratio of HSA-DBCO and Uox was highest when reacting in a molar ratio of 2:1.

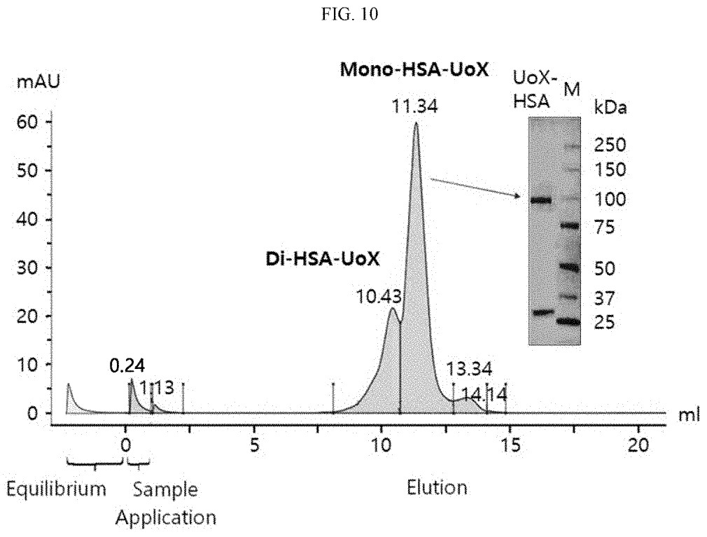

As confirmed in FIG. 10, a new band having a size of approximately 101 kDa was able to be detected by SDS-PAGE.

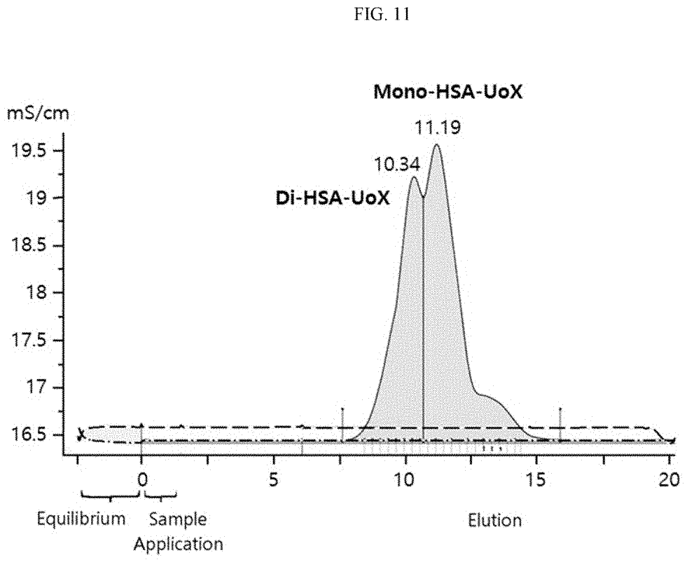

As confirmed in FIG. 11, as the molar ration increases, the proportion of di-HSA-Uox is higher than that of mono-HSA-Uox.

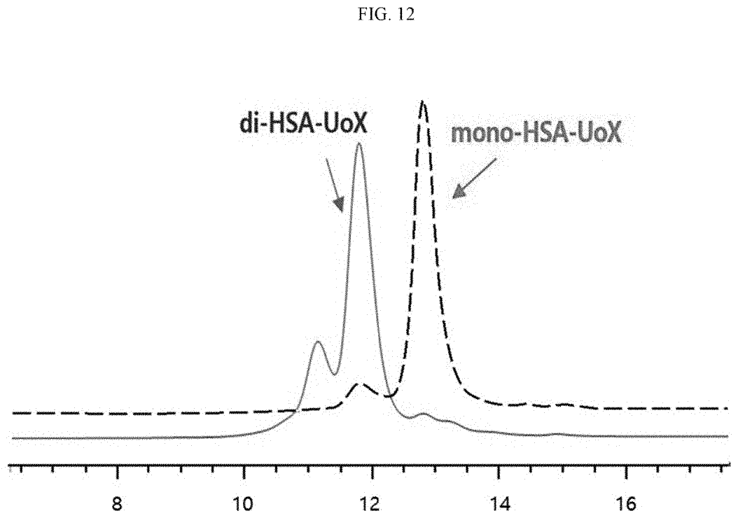

As confirmed in FIG. 12, the separation of mono-HSA-Uox and di-HSA-Uox was confirmed by SEC-HPLC.

### Example 6. Measurement of Enzyme Activity of UoX-HSA

To analyze the enzyme activity of Uox-HSA, spectroscopy was used. A decreasing concentration of uric acid over time when 100 μM uric acid was reacted with 60 nM Uox-HSA was analyzed using a Ultrospec 2100 pro UV/Visible spectrophotometer (Biochrom, Cambridge, UK) at 293 nm, which is the maximum absorbance wavelength of uric acid. The unit (U/mL) of enzyme activity was obtained by multiplying an absorbance change (Δ_{ABS293nm}/min) by the total reaction volume, dividing by the molar absorption coefficient of uric acid (12.3 mM⁻¹ cm⁻¹), and then dividing by the volume of an enzyme. The unit (U/mL) of enzyme activity was defined as the amount of an enzyme capable of converting 1 μM of uric acid into allantoin per minute at room temperature. An activity per unit mass (specific activity, U/mg) of the enzyme was obtained by dividing by an amount of the enzyme used in the reaction.

The result is shown in Table 2.

TABLE 2
Uox-HSA enzyme activity

| | M.W. (kDA) | U/mL | U/mg |
|---|---|---|---|
| Uox-WT | 136 | 0.38 ± 0.002 | 46.14 ± 0.194 |
| Mono-HSA-Uox | 203 | 0.35 ± 0.008 | 28.61 ± 0.663 |
| Di-HSA-Uox | 270 | 0.37 ± 0.043 | 22.63 ± 2.65 |

As confirmed in Table 2, the enzyme activities of Uox-WT, mono-HSA-Uox and di-HSA-Uox were measured to be 0.38 U/mL, 0.35 U/mL and 0.37 U/mL, respectively. This confirmed that the albumin conjugation does not affect the enzyme activity of Uox, and the Uox-HSA conjugate maintains activity even with one or two albumin bindings. A difference in the enzyme activity (U/mg) per unit mass is caused by an increase in molecular weight of the albumin-conjugated Uox-HSA conjugate.

It was confirmed that di-HSA-Uox in which two albumins are conjugated has greater enzyme activity than mono-HSA-Uox in which one albumin is conjugated.

### Example 7. Confirmation of Uric Acid Level Reducing Effect of UoX-HSA Using Gout-Induced Animal Model

An experiment for the uric acid level reducing effect of Uox-HSA using a gout-induced animal model was carried out with mice and rats.

As experimental animals, 8 to 10-week-old male C57BL mice (20 to 25 g, Samtako) bred in an environment maintained at a humidity of 50±5% and a temperature of 24 to 26° C. were used for a subsequent experiment after acclimating in a laboratory environment for 1 week with sufficient supply of feed and water. The induction of hyperuricemia was performed by dissolving hypoxanthine, which is a uric acid precursor, in 1 mL of a 3% starch solution and then orally administering it at a concentration of 500 mg/kg, and 10 minutes after administration, dissolving potassium oxonate, which is an Uox inhibitor, in 1 mL of 0.5% sodium carboxymethylcellulose and then intraperitoneally injecting it at a concentration of 250 mg/kg. Allopurinol used as a control and a uric acid-forming inhibitor was orally administered at 50 mg/kg 10 minutes after hypoxanthine and potassium oxonate were intraperitoneally injected. Uox-WT (Rasburicase) and Uox-HSA were intravenously administered at 3.4 mg/kg and 5.0 mg/kg, respectively, to evaluate whether a uric acid level in blood decreases over time. An experimental group was administered Uox-HSA according to an administration route and an administration dose as shown in Table 3 below, and blood was sampled from a caudal vein over time. The uric acid level in blood was quantified by an FRAP method. 30 uL of blood and 300 uL of a working solution (10 mM TPTZ, 20 mM FeCl₃, 300 mM Acetate buffer) were mixed, and absorbance was measured at 593 nm.

As experimental animals, 7-week-old male Sprague-Dawley rats (250 to 280 g, Samtako) bred under an environment maintained at a humidity of 50±5% and a temperature of 24 to 26° C. were used for a subsequent experiment after acclimating in a laboratory environment for 1 week with sufficient supply of feed and water. The induction of hyperuricemia was performed by dissolving hypoxanthine, which is a uric acid precursor, in 1 mL of 3% starch solution, and one hour after administration, dissolving potassium oxonate, which is an Uox inhibitor, in 1 mL of 0.5% sodium carboxymethylcellulose and then orally administering it at a concentration of 250 mg/kg for 7 days. Allopurinol used as a control and a uric acid-forming inhibitor was administered for 7 days with the hyperuricemia induction to inhibit the formation of uric acid. Uox-WT (Rasburicase) and Uox-HSA were intravenously administered at 3.4 mg/kg and 5.0 mg/kg, respectively, to evaluate whether a uric acid level in blood decreases over time. An experimental group was administered with Uox-HSA according to an administration route and an administration dose as shown in Table 4 below, and blood was collected from a caudal vein over time, centrifuged at 3,000 rpm for 10 minutes to separate plasma, followed by quantification using a uric acid assay kit (Abnova, Taipei, Taiwan).

TABLE 3
Group classification and dose of mouse animal models

| | | | Dose | No. of |
|---|---|---|---|---|
| Group | Drug | Route | (mg/kg) | animals |
| 1 | Control | — | — | 3 |
| 2 | Hyperurecemia + PBS | I.V. | — | 3 |
| 3 | Hyperurecemia + Uox-WT | I.V. | 3.4 | 3 |
| 4 | Hyperurecemia + Uox-HSA | I.V. | 5.0 | 3 |
| 5 | Hyperurecemia + Allopurinol | Oral | 50 | 3 |

TABLE 4
Group classification and dose of rat animal models

| | | | Dose | No. of |
|---|---|---|---|---|
| Group | Drug | Route | (mg/kg) | animals |
| 1 | Control | — | — | 3 |
| 2 | Hyperurecemia | I.V. | — | 6 |
| 3 | Hyperurecemia + Uox-WT | I.V. | 3.4 | 6 |
| 4 | Hyperurecemia + Uox-HSA | I.V. | 5.0 | 6 |
| 5 | Hyperurecemia + Allopurinol | Oral | 50 | 4 |

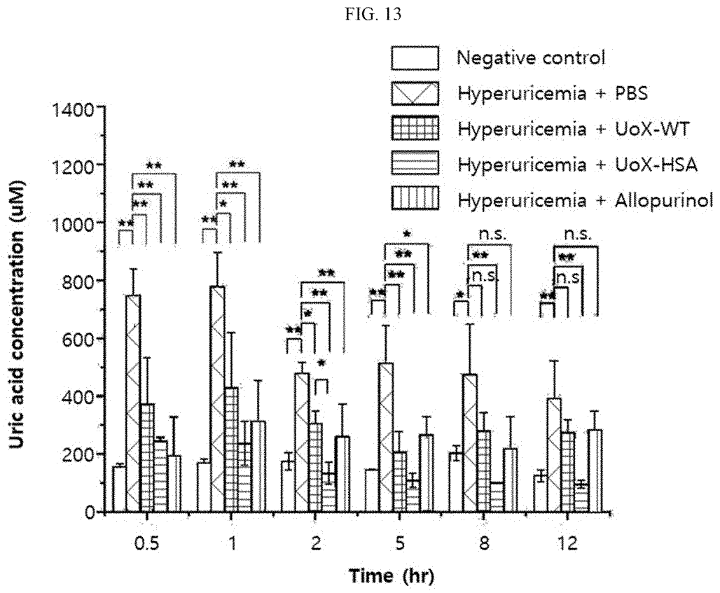

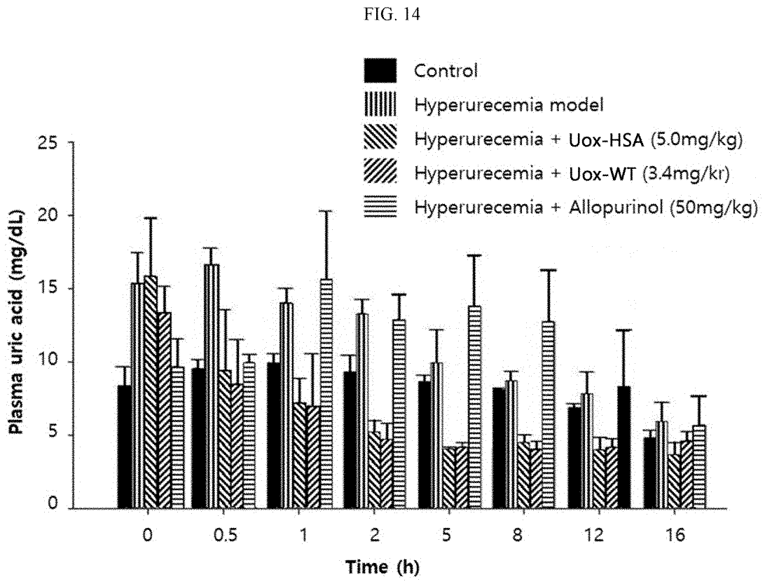

The results are shown in FIGS. 13 and 14.

As confirmed in FIG. 13, the uric acid level in blood began to rapidly decrease 30 minutes to 2 hours after administration of Uox-HSA in a mouse animal model and was maintained for 12 hours. Uox-WT had a decreased uric acid level in blood until 5 hours, but a uric acid level began to be increase after 5 hours.

As confirmed in FIG. 14, the uric acid in blood began to decrease from 30 minutes and then was maintained until 16 hours after administration of Uox-HSA in rat animal models. This means that the activity of Uox-HSA tends to be maintained and its persistency also increases, compared with Uox-WT (Rasburicase), which is a conventional drug. On the other hand, after administration of the uric acid-forming inhibitor, allopurinol, used as a control, a uric acid level began to decrease until 30 minutes, and began to increase 1 hour after administration.

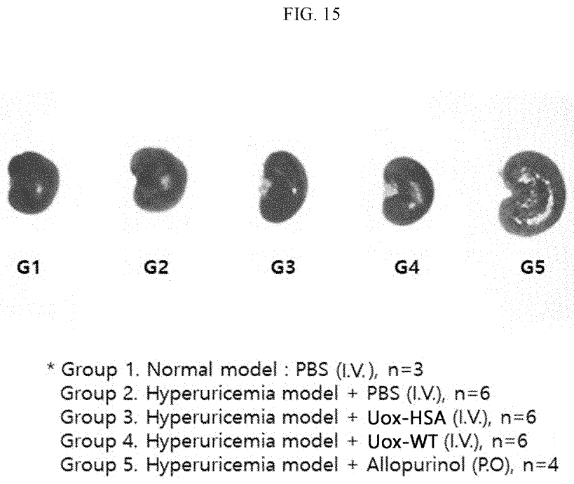

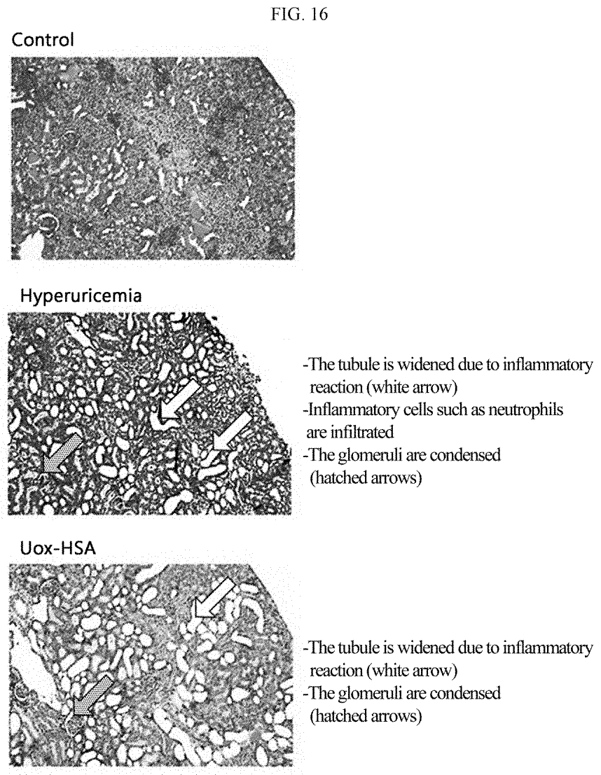

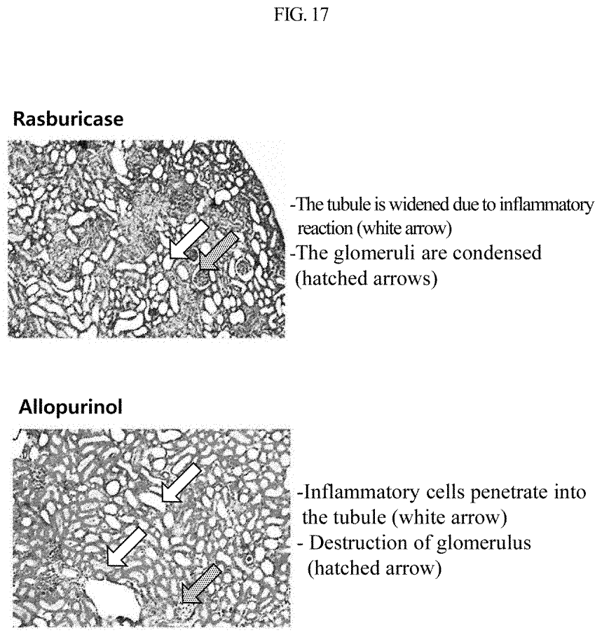

In addition, as confirmed in FIGS. 15 to 17, as a result of kidney autopsy, a severe renal damage was observed by allopurinol administration, compared with the other groups.

### Example 8: Separation and Purification Process for AzF-Introduced UoX-2

To separate and purify Uox from the pellet harvested from the process described up to Example 2, 3-step chromatography was performed. The pellet was suspended in a suspension buffer (20 mM Tris-HCl pH 9.0), 1 mg/mL of lysozyme was added, followed by dissolution for 30 minutes. Only a supernatant was obtained by centrifugation at 10,000 rpm for 20 minutes, and then purified by 3-step chromatography. In the first step, elution was performed with a 0.5M NaCl gradient method using an anion exchange column (Hitrap DEAE FF, GE Healthcare). In the second step, following equilibration with a 0.5 M (NH₄)₂SO₄-containing solvent through hydrophobic interaction chromatography using a hydrophobic column (Hitrap phenyl(High sub), GE Healthcare), a sample was injected, and then eluted with a 20 mM Tris-HCl pH 9.0 solvent. Subsequently, to increase purity, 3-step size exclusion chromatography was carried out, separation was performed using a size exclusion column (Superdex 200 10/300 GL), and then the purity was confirmed by analysis with SDS-PAGE and SEC-HPLC (AdvanceBio SEC 300A, 7.8×300 mm, 2.7 μm). For a comparative experiment, E. Coli Top10 (pQE80-Uox-WT) was cultured and separated by the same method except a process of injecting AzF to produce Uox-WT.

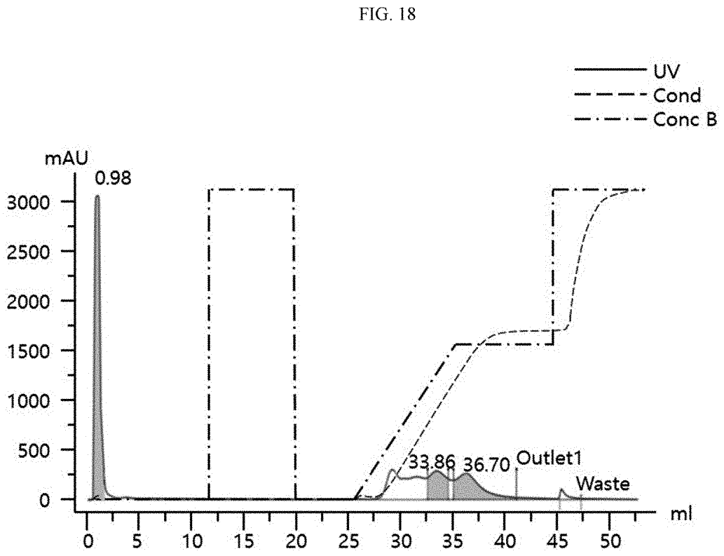

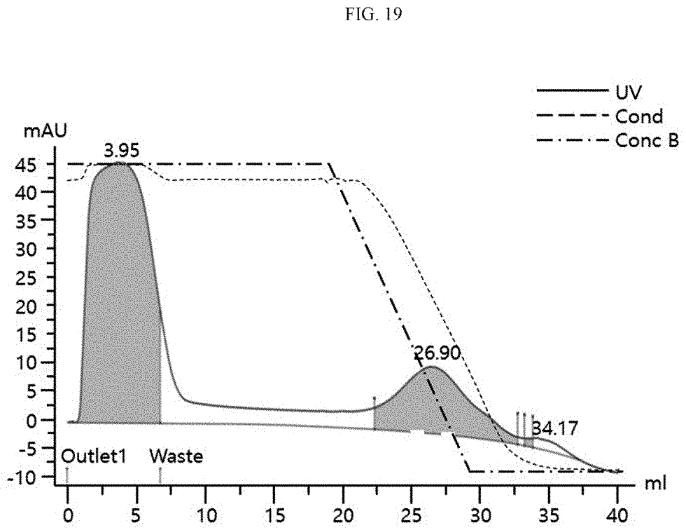

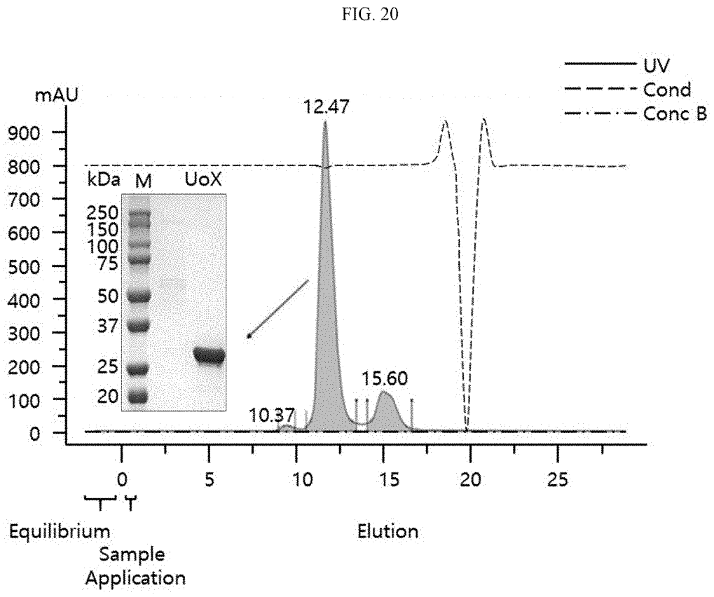

The results are shown in FIGS. 18 to 21.

As confirmed in FIGS. 18 to 20, the total yield of Uox was 1 to 5 mg/g.

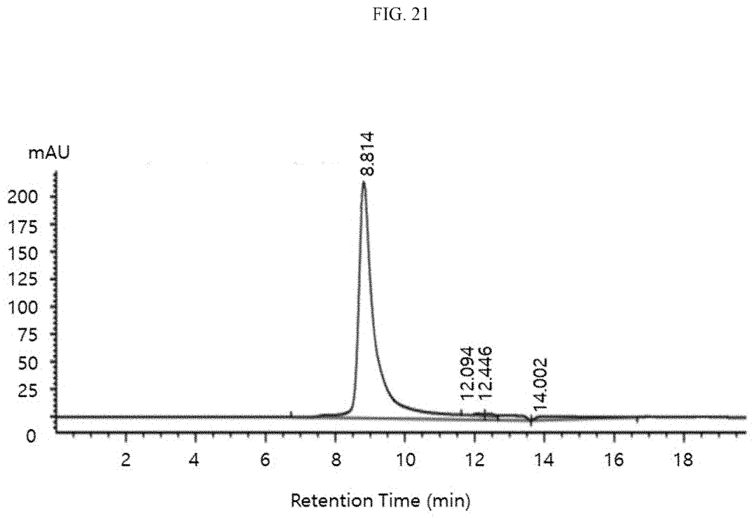

As confirmed in FIG. 21, as a result of SEC-HPLC, Uox with a purity of 95% was separated.

### Example 9: Analysis of Molecular Weight of Di-HSA-UoX Through SEC-LC/MS

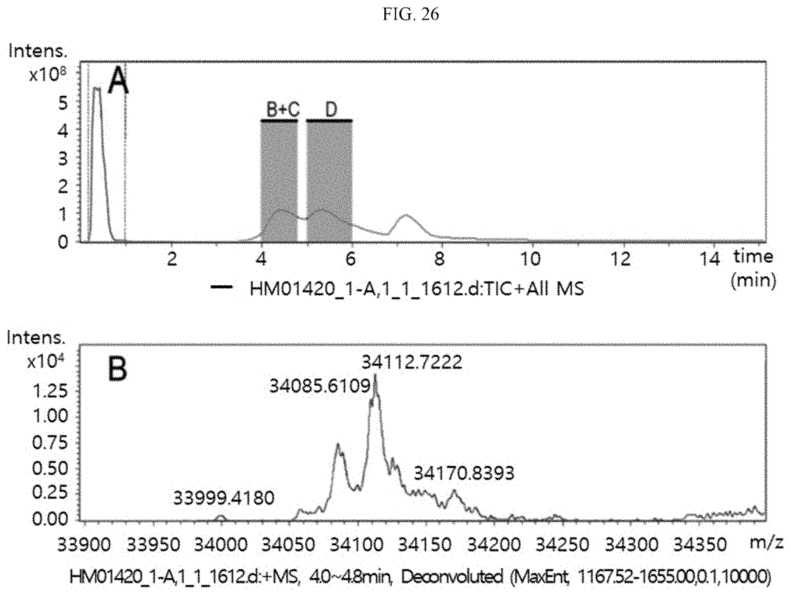

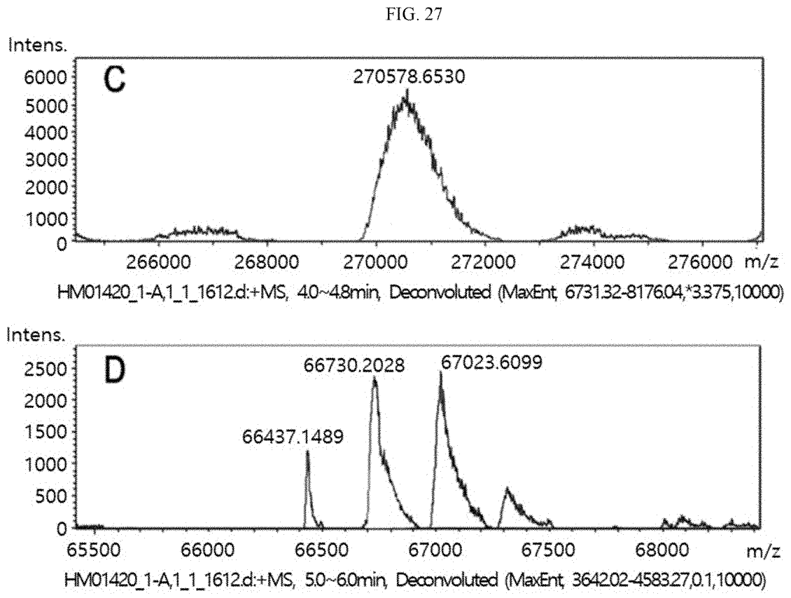

The results measured through an experiment of measuring the molecular weight of Di-HSA-Uox are shown in Table 5 and FIGS. 26 and 27 below.

TABLE 5
Result of confirmation of molecular weight of Uox0di-HSA conjugate

| | Uox-di-rUSA conjugate (PS-P23-2020-0002) | | | |
|---|---|---|---|---|
| | Experimental | Theoretical | | |
| | mass | mass | | mass |
| Sample | (average) | (average) | Possible | difference |
| File name | [Da] | [Da] | compounds | [Da] |
| HM01420 | 34112.7 | 34111.1 | Urate oxidase | 1.6 |
| | | | (2× AzF: without | |
| | | | N-terminal | |
| | | | methionine) | |
| | 270578.7 | 270668.6 | Tetramer | −89.9 |
| | | | (4× Uox + 2× | |
| | | | Linker + 2× rHSA) | |
| | 66437.3 | 66437.1 | Human Serum | 0 . . . 2 |
| | | | Albumin | |
| | | | (rHSA) | |

In FIGS. 26 and 27, A is a total ion chromatogram (TIC) result, B is a separated mass spectrum of Uox (2×AZF, without N-terminal methionine), C is a separated mass spectrum of Uox-HSA (4× Uox+2× Linker+2× rHSA), and D is a separated mass spectrum of HSA.

As confirmed in FIGS. 26 and 27, the molecular weight of Uox-HSA was measured to be 270578.7 Da, similar to the theoretical value of di-HSA-Uox (270668.6 Da).

### Example 10: Analysis of Isoelectric Point (pI) Characteristic of UoX-HSA Using cIEF

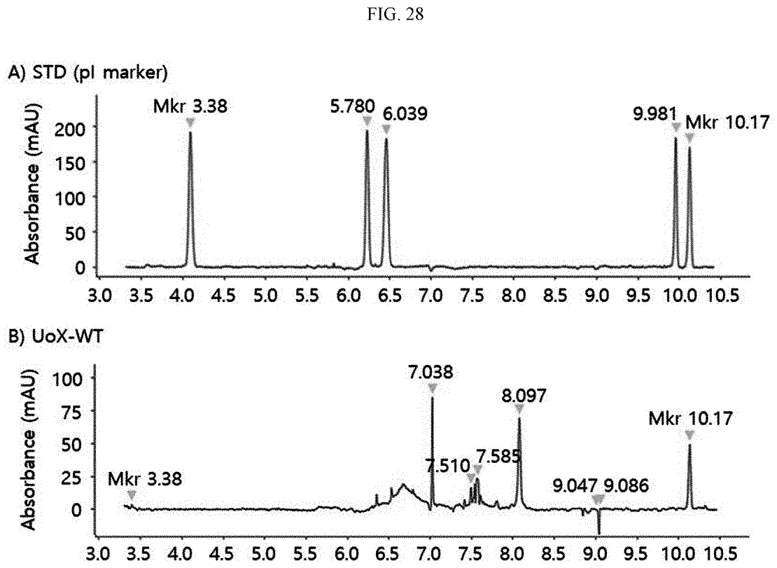

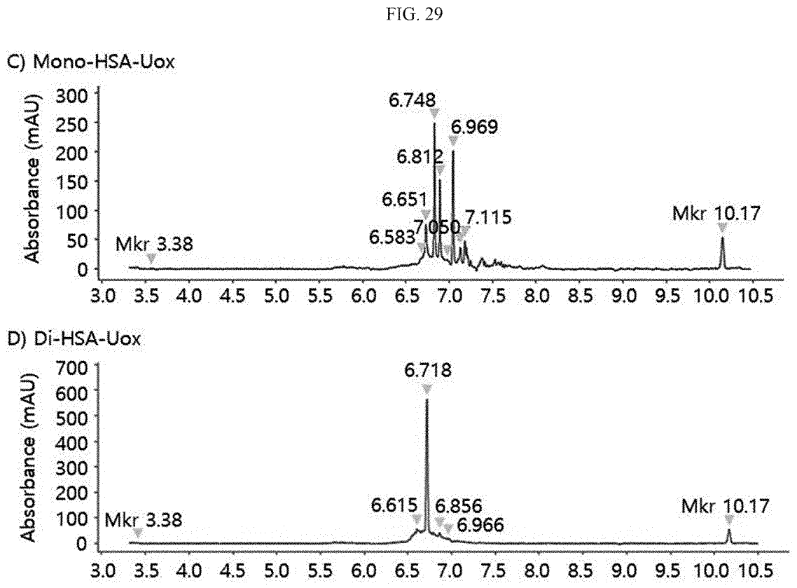

cIEF analysis was carried out for pI analysis of separated and purified Uox-HSA. As analysis equipment, ProteinSimple Maurice was used. The pI of Uox-WT, mono-HSA-Uox and di-HSA-Uox was assessed using a pH 3 to 10 separation range (pI marker). The analysis results are shown in FIGS. 28 and 29.

As confirmed in FIGS. 28 and 29, Di-HSA-Uox was analyzed as a main peak at pI 6.72, and Uox-WT was detected with various peaks in a PI range from 7.04 to 9.09. Mono-HSA-Uox was detected with various peaks in a pI range from 6.58 to 7.51.

### Example 11: Measurement of In Vitro Enzyme Activity of UoX-HSA

Unless described otherwise, Uox-HSA described in the following example refers to di-HSA-Uox. To analyze the enzyme activity of Uox-HSA, spectroscopy was used. A decreasing concentration of uric acid over time when 100 μM uric acid was reacted with 60 nM Uox-HSA was analyzed using a Hidex Microplate reader (Hidex, Finland) at 293 nm, which is the maximum absorbance wavelength of uric acid. The unit (U/mL) of enzyme activity was obtained by multiplying an absorbance change (Δ_{ABS293nm}/min) by the total reaction volume, dividing by the molar absorption coefficient of uric acid (12.3 mM⁻¹ cm⁻¹), and then dividing by the volume of an enzyme. The unit (U/mL) of enzyme activity was defined as the amount of an enzyme capable of converting 1 μM of uric acid into allantoin per minute at room temperature. An activity per unit mass (specific activity, U/mg) of the enzyme was obtained by dividing by an amount of the enzyme used in the reaction.

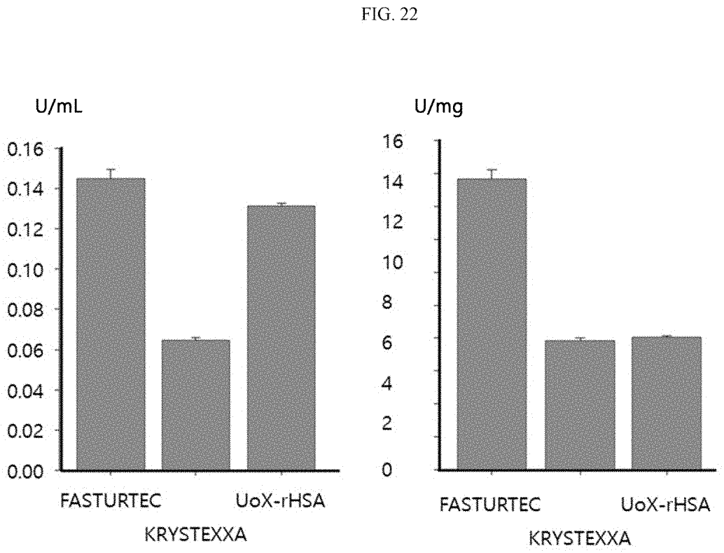

The result is shown in Table 6 and FIG. 22.

TABLE 6
UoX-HSA enzyme activity

| | U/mL | U/mg |
|---|---|---|
| Uox-HSA | 0.13 | 8.1 |
| FASTURTEC | 0.14 | 17.6 |
| KRYSTEXXA | 0.06 | 7.9 |

As confirmed in Table 6, the enzyme activities per unit dose of Uox-HSA, FASTURTEC and KYRSTEXXA were measured to be 0.13 U/mL, 0.14 U/mL and 0.06 U/mL, respectively. Particularly, the activities of Uox-HSA and KRYSTEXXA showed a 2-fold or more difference. The difference between an enzyme activity per unit mass (U/mg) and an enzyme activity per unit dose of the Uox-HSA conjugate is due to an increase in molecular weight of the albumin-conjugated Uox-HSA conjugate. It was possible to confirm that, compared with KRYSTEXXA, the same amount of Uox-HSA has significantly improved activity.

### Example 12: Uric Acid Level Reducing Effect of UoX-HSA Using Animal Model in which Gout was Repeatedly Induced

As experimental animals, 6-week-old male Sparague-Dawley rats (190 to 210 g, Samtako) bred under an environment maintained at a humidity of 50±5% and a temperature of 22±3° C. were used for a subsequent experiment after acclimating in a laboratory environment for 1 week with sufficient supply of feed and water. The repeated induction of hyperuricemia was carried out by dissolving a uric acid precursor, hypoxanthine, in 1 mL of a 3% starch solution and then orally administering it at a concentration of 500 mg/kg, and 10 minutes after administration, dissolving an Uox inhibitor, potassium oxonate, in 1 mL of 0.5% sodium carboxymethylcellulose and then intraperitoneally administering it at a concentration of 250 mg/kg. The repeated induction was performed a total of 4 times: two days before administration of a test material, one day before administration of a test material, on the day of administration and one day after administration. This experiment was performed to confirm the persistency of the test drug by reinducing hyperuricemia one day after administration of the test drug unlike Example 7.

As an administration dose of the test drug, Uox-HSA was intravenously administered at 2.0 mg/kg to evaluate whether a uric acid level in blood was reduced over time. As a control drug, FASTURTEC (Sanofi-Aventis) and the only persistent gout therapeutic, KRYSTEXXA (Horizon Pharma), were intravenously administered at 2.0 mg/kg to evaluate whether a uric acid level in blood was reduced over time (Table 7). The uric acid level in blood was quantified using a uric acid assay kit (Abnova, Taipei, Taiwan) after plasma was separated by centrifugation of the blood collected from a caudal vein.

TABLE 7
Conditions for administration of test drug

| | | | Dose | Dose | No. of |
|---|---|---|---|---|---|
| Group | Drug | Route | (mg/kg) | (nmol/kg) | animals |
| 1 | Hyperurecemia | I.V. | — | — | 4 |
| 2 | Hyperurecemia + | I.V. | 2.0 | 7.4 | 4 |
| | Uox-HSA | | | | |
| 3 | Hyperurecemia + | I.V. | 2.0 | 14.6 | 4 |
| | FASTURTEC | | | | |
| 4 | Hyperurecemia + | I.V. | 2.0 | 14.6 | 4 |
| | KRYSTEXXA | | | | |

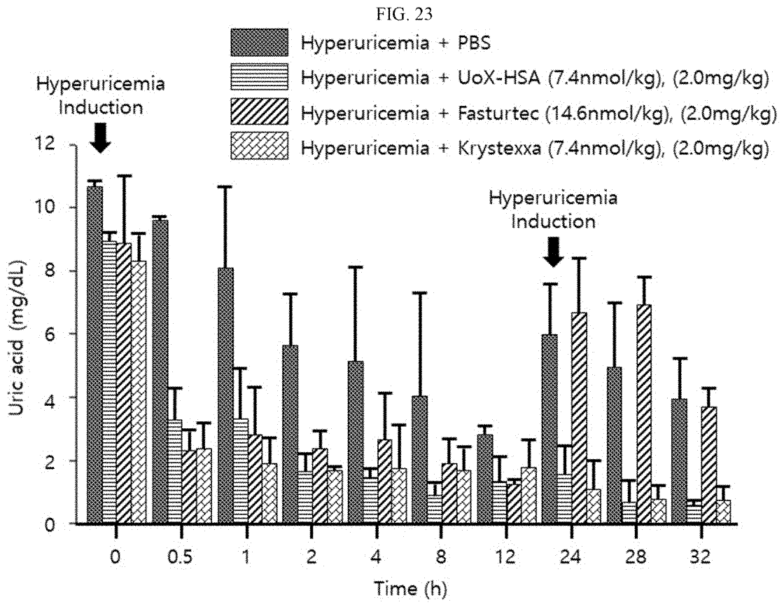

As confirmed in FIG. 23, in all of the Uox-HSA, FASTURTEC and KRYSTEXXA-administered groups of the repeated induction animal models, the uric acid level in blood began to decrease after 30 minutes and was maintained until 12 hours. Afterward, as a result of reinduction at 24 hours, FASTURTEC did not show an effect of reducing uric acid, whereas Uox-HSA and KRYSTEXXA showed an effect of reducing uric acid to a normal level or less. In addition, Uox-HSA showed an equal or higher effect of reducing uric acid to that of KRYSTEXXA with only half the amount. From this, it was possible to confirm that Uox-HSA is a drug having high persistency and excellent activity compared with the conventional Uox drugs.

### Example 13: Evaluation of Pharmacodynamic Characteristic in SD-Rt Model

As experimental animals, 5-week-old male Sparague-Dawley rats (190 to 210 g, Orient Bio) bred in an environment maintained at a humidity of 50±5% and a temperature of 22±3° C. were used for a subsequent experiment after acclimating in a laboratory environment for 1 week with sufficient supply of feed and water.

As an administration dose of the test drug, Uox-HSA was intravenously administered at 4.0 mg/kg to evaluate whether a uric acid level in blood was reduced over time. As a control drug, FASTURTEC (Sanofi-Aventis) and the only persistent gout therapeutic, KRYSTEXXA (Horizon Pharma), were intravenously administered at 2.0 mg/kg to evaluate whether a uric acid level in blood was reduced over time. Blood was collected form a caudal vein, centrifuged to separate plasma, followed by analysis by a Uricase activity assay method.

TABLE 8
Conditions for administration of test drug

| | | | | Dose | No. of |
|---|---|---|---|---|---|
| Group | Drug | Route | Dose (mg/kg) | (nmol/kg) | animals |
| 1 | UoX-HSA | I.V. | 4.0 | 14.6 | 5 |
| 2 | FASTURTEC | I.V. | 2.0 | 14.6 | 5 |
| 3 | KRYSTEXXA | I.V. | 2.0 | 14.6 | 5 |

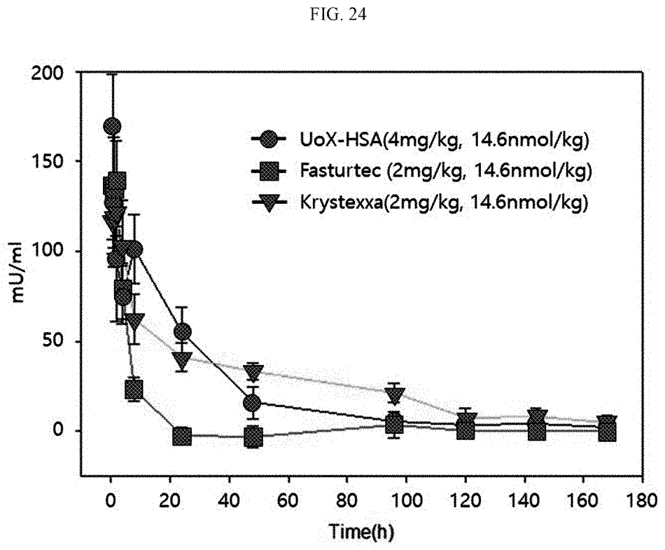

As confirmed in FIG. 24 and Table 9 below, FASTURTEC showed a short half-life of 2.1 hours, whereas Uox-HSA showed a half-life of 27 hours, which was approximately 8.7-fold higher than that of FASTURTEC. The persistent gout therapeutic, KRYSTEXXA, showed a half-life of 35.2 hours. The size of KRYSTEXXA is approximately 500 kDa or more so that it is not filtered in the kidney and thus keeps maintaining its activity. Uox-HSA includes two human-derived albumins bound thereto, and the half-life is expected to be more prolonged due to recycle of FcRn recycling by albumin in clinical trials.

TABLE 9
Result of measurement of half-life of test drug

| | t½ (h) | AUC (mU/mL × h) | |
|---|---|---|---|
| Uox-HSA | 27.0 | 3,739.5 | From 0.5 to 168 h |
| FASTURTEC | 3.1 | 788.2 | From 0.5 to 24 h |
| KRYSTEXXA | 35.2 | 5,456.0 | From 0.5 to 168 h |

### Example 14: Comparison of Pharmacodynamic Characteristics of Di-HSA-UoX and Mono-HSA-UoX

As experimental animals, 7-week-old male Sparague-Dawley rats (250 to −280 g, Samtako) bred in an environment maintained at a humidity of 50±5% and a temperature of 24 to 26° C. were used for a subsequent experiment after acclimating in a laboratory environment for 1 week with sufficient supply of feed and water. Mono-HSA-Uox and di-HSA-Uox were intravenously administered at 5.0 mg/kg and 6.6 mg/kg, respectively, and then blood samples were collected 0, 0.5, 1, 2, 4, 8, 24, 48 and 96 hours after administration to analyze enzyme activity.

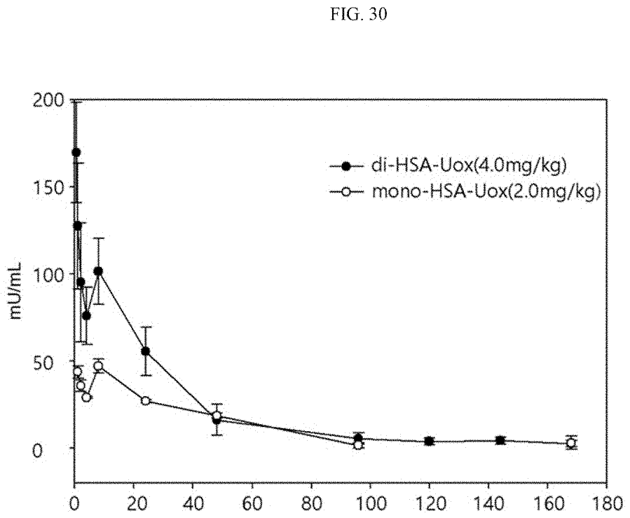

The result of comparing the characteristic of di-HSA-Uox measured in Example 13 and the characteristic of mono-HSA-Uox measured in this example are shown in Table 10 and FIG. 30.

TABLE 10
Comparison of pharmcodynamic characteristics of
di-HAS-Uox and mono-HSA-Uox

| | t½ (h) | AUC (mU/mL × h) |
|---|---|---|
| Di-HSA-Uox | 27.0 | 3739.5 |
| Mono-HSA-Uox | 21.8 | 1243.7 |

As confirmed in Table 10, it was able to be confirmed that di-HSA-Uox has a half-life approximately 24% longer than mono-HSA-Uox. This shows that, as expected in the section 5.4., when an albumin binding number increases, an effect of improving half-life is exhibited.

### Example 15: Immunogenic Analysis in CD-1 Mouse by 4-Week Repetitive Administration

As experimental animals, 5-week-old female Hsd:ICR (CD-1) mice (20 to 25 g, Orient Bio) bred in an environment maintained at a humidity of 50±5% and a temperature of 22±3° C. were used for a subsequent experiment after acclimating in a laboratory environment for 1 week with sufficient supply of feed and water.

As an administration dose of the test drug, Uox-HSA was injected into a thigh muscle at 4.0 mg/kg, and administered once a week for five weeks. As a control drug, FASTURTEC was injected into a thigh muscle at 2.0 mg/kg, and administered once a day for 7 days (Table 11). Blood collected every week for 6 weeks was centrifuged to separate plasma, followed by analysis using an anti-uricase antibody ELISA method.

TABLE 11
Conditions for administration of test
drug for immunogenicity test

| Group | Drug | Route | Dose (mg/kg) | No. of animals |
|---|---|---|---|---|
| 1 | Uox-HSA | I.M. | 4.0 | 4 |
| 2 | FASTURTEC | I.M. | 2.0 | 4 |

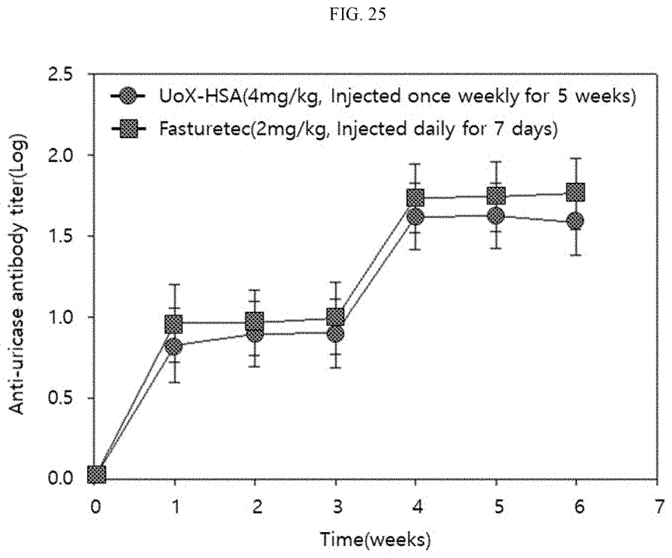

As confirmed in FIG. 25 and Table 11, it was possible to confirm that the Uox-HSA-administered group shows slightly less formation of anti-uricase antibodies compared with the FASTURTEC-administered group. It was possible to confirm that the immunogenicity of the foreign material such as Uox is reduced by the conjugation of HSA. Further, since the HSA is human albumin, when being actually administered to a human, it is expected that a more improved immunogenic reducing effect will be exhibited.

Regarding the above description, it should be understood by those of ordinary skill in the art that the above descriptions of the present application are exemplary, and the exemplary embodiments disclosed herein can be easily modified into other specific forms without changing the technical spirit or essential features of the present application. Therefore, it should be interpreted that the exemplary embodiments described above are exemplary in all aspects, and are not limitative. The scope of the present application is defined by the appended claims and encompasses all modifications and alterations derived from meanings, the scope and equivalents of the appended claims.

1. A Uox-carrier conjugate of formula 1: 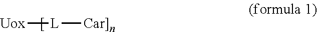
wherein UoX is a uricase from Aspergillus Flavus formed from 4 uricase monomers (p′);
L is a crosslinker; and
Car is a carrier,
wherein the p′ has an amino acid sequence of SEQ ID NO:1 substituted at at least one amino acid residue selected from the group of tyrosine 8, tyrosine 16, tyrosine 30, tyrosine 46, tyrosine 65, phenylalanine 79, phenylalanine 87, tyrosine 91, tryptophan 106, phenylalanine 120, phenylalanine 159, tryptophan 160, phenylalanine 162, tyrosine 167, tryptophan 174, tryptophan 186, tryptophan 188, phenylalanine 191, phenylalanine 204, tryptophan 208, phenylalanine 219, tyrosine 233, tyrosine 251, tyrosine 258, phenylalanine 259, tryptophan 265, and phenylalanine 279 by a non-natural amino acid selected from p-Azido-L-phenylalanine (AzF), p-ethynyl-phenylalanine (pEthF), L-Homopropargylglycine (HPG), O-propargyl-L-tyrosine (oPa) and p-propargyloxyphenylalanine (pPa),
the Uox and the L is linked through the substituted non-natural amino acid,
and n=2.

2. The Uox-carrier conjugate according to claim 1,
wherein the Car is an albumin.

3. The Uox-carrier conjugate according to claim 1,
wherein the p′ has an amino acid sequence of SEQ ID NO:1 substituted at tryptophan 160, or tryptophan 174 by a non-natural amino acid selected from p-Azido-L-phenylalanine (AzF), p-ethynyl-phenylalanine (pEthF), L-Homopropargylglycine (HPG), O-propargyl-L-tyrosine (oPa) and p-propargyloxyphenylalanine (pPa).

4. The Uox-carrier conjugate according to claim 1,
wherein the substituted non-natural amino acid is p-Azido-L-phenylalanine (AzF).

5. The Uox-carrier conjugate according to claim 1,
wherein the L comprises alkylene, alkenylene, alkynylene, or (CH2O)n.

6. The Uox-carrier conjugate according to claim 5,
wherein the L comprises (CH2O)₁₀.

7. The Uox-carrier conjugate according to claim 2,
wherein the albumin is a human serum albumin.

8. The Uox-carrier conjugate according to claim 1,
wherein the linked structure of the Uox and the L is a bonding structure formed by a click-chemistry reaction.

9. The Uox-carrier conjugate according to claim 8,
wherein the linked structure of the Uox and the L is a bonding structure formed by a reaction between dibenzocyclooctyne (DBCO) and azide.

10. The Uox-carrier conjugate according to claim 2,
wherein the linked structure of the L and the albumin is a bonding structure formed by a reaction between maleimide (MAL) and a cysteine residue of the albumin.

11. The Uox-carrier conjugate according to claim 1,
wherein the Uox is formed from two p′ each of which is not linked to the carrier and two p′ each of which is linked to the carrier.

12. The method for manufacturing the Uox-carrier conjugate according to claim 1, comprising
producing a uricase comprising a non-natural amino acid by culturing bacteria by fed-batch culture;
separating the uricase comprising a non-natural amino acid;
conjugating the uricase comprising a non-natural amino acid with a carrier; and
separating the uricase conjugated to the carrier,
wherein the separating is performing a hydrophobic interaction chromatography, an anion exchange chromatography, and a size exclusion chromatography.

13. The method for manufacturing the Uox-carrier conjugate according to claim 12,
wherein the separating the uricase comprising a non-natural amino acid; is performing a hydrophobic interaction chromatography, an anion exchange chromatography, and a size exclusion chromatography in order.

14. The method for manufacturing the Uox-carrier conjugate according to claim 12,
wherein the separating the uricase comprising a non-natural amino acid; is performing an anion exchange chromatography, a hydrophobic interaction chromatography, and a size exclusion chromatography in order.

15. A composition for preventing or treating at least one disease selected from the group of hyperuricemia, gout, deposition of urate crystals in joints, acute gouty arthritis by deposition of urate crystals in joints, urolithiasis, nephrolithiasis, and gouty nephropathy.

16. A food composition for preventing or ameliorating at least one disease selected from the group of hyperuricemia, gout, deposition of urate crystals in joints, acute gouty arthritis by deposition of urate crystals in joints, urolithiasis, nephrolithiasis, and gouty nephropathy.


SEQUENCE LISTING (12 sequences)

SEQ ID NO: 1 - protein, 301 aa, Artificial Sequence
  Aspergillus flavus_Uox
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 2 - protein, 301 aa, Artificial Sequence; identical to SEQ ID NO: 1
  Aspergillus flavus_Uox
  VARIANT (160)..(160): p-Azido-L-phenylalanine

SEQ ID NO: 3 - protein, 301 aa, Artificial Sequence; identical to SEQ ID NO: 1
  Aspergillus flavus_Uox
  VARIANT (174)..(174): p-Azido-L-phenylalanine

SEQ ID NO: 4 - protein, 301 aa, Artificial Sequence; identical to SEQ ID NO: 1
  Aspergillus flavus_Uox
  VARIANT (160)..(160): p-Azido-L-phenylalanine
  VARIANT (174)..(174): p-Azido-L-phenylalanine

SEQ ID NO: 5 - DNA, 927 bp, Artificial Sequence
  Aspergillus flavus_Uox
atgtctgctgtgaaggccgcaagatatggcaaggataatgtgagggtgtacaaggtgcataaggacgaaaagactggcgtgcagacagtgtacgagatgaccgtgtgcgtcctgctggagggcgaaatcgagacttcttataccaaagccgacaactccgtgattgtggccacagattctatcaagaacactatctatatcaccgccaaacagaacccagtgacaccacctgaactgttcggcagcattctcggcacacactttattgagaagtacaaccacatccatgctgcacacgtgaatatcgtgtgtcatcgctggacccgcatggacatcgacggaaagccacacccccactcttttatcagagactctgaagaaaagagaaacgtgcaggtcgacgtggtggagggaaaaggtatcgacatcaagagctcactctccggcctgaccgtgctgaagagtaccaattcacagttttgggggtttctgagagacgaatacactacactgaaggagacttgggatagaatcctgagtaccgacgtggatgcaacctggcagtggaagaatttttccgggctgcaggaagtgcggtcccacgtgcccaagtttgatgcaacctgggcaaccgcaagggaggtgacactgaaaacctttgccgaggacaactccgctagcgtgcaggccacaatgtacaagatggccgaacagatcctggccagacagcagctgattgagactgtggagtactctctgcctaacaagcactatttcgaaatcgacctgtcctggcacaagggactgcagaatactggtaaaaacgcagaggtgttcgcccctcagagtgatcccaatggtctgatcaaatgcacagtggggagatcctctctgaagagcaagctgcaccatcaccatcaccattaa

SEQ ID NO: 6 - DNA, 38 bp, Artificial Sequence
  forward primer for his tag deletion vector
ctctctgaagagcaagctgtaagcttaattagctgagc

SEQ ID NO: 7 - DNA, 38 bp, Artificial Sequence
  reverse primer for his tag deletion vector
gctcagctaattaagcttacagcttgctcttcagagag

SEQ ID NO: 8 - DNA, 27 bp, Artificial Sequence
  primer for amb 160
caattcacagttttaggggtttctgag

SEQ ID NO: 9 - DNA, 27 bp, Artificial Sequence
  primer for amb 160
ctcagaaacccctaaaactgtgaattg

SEQ ID NO: 10 - DNA, 29 bp, Artificial Sequence
  primer for amb 174
cactgaaggagacttaggatagaatcctg

SEQ ID NO: 11 - DNA, 29 bp, Artificial Sequence
  primer for amb 174
caggattctatcctaagtctccttcagtg

SEQ ID NO: 12 - DNA, 903 bp, Artificial Sequence
  amb variant UoX
tctgctgtgaaggccgcaagatatggcaaggataatgtgagggtgtacaaggtgcataaggacgaaaagactggcgtgcagacagtgtacgagatgaccgtgtgcgtcctgctggagggcgaaatcgagacttcttataccaaagccgacaactccgtgattgtggccacagattctatcaagaacactatctatatcaccgccaaacagaacccagtgacaccacctgaactgttcggcagcattctcggcacacactttattgagaagtacaaccacatccatgctgcacacgtgaatatcgtgtgtcatcgctggacccgcatggacatcgacggaaagccacacccccactcttttatcagagactctgaagaaaagagaaacgtgcaggtcgacgtggtggagggaaaaggtatcgacatcaagagctcactctccggcctgaccgtgctgaagagtaccaattcacagttttaggggtttctgagagacgaatacactacactgaaggagacttaggatagaatcctgagtaccgacgtggatgcaacctggcagtggaagaatttttccgggctgcaggaagtgcggtcccacgtgcccaagtttgatgcaacctgggcaaccgcaagggaggtgacactgaaaacctttgccgaggacaactccgctagcgtgcaggccacaatgtacaagatggccgaacagatcctggccagacagcagctgattgagactgtggagtactctctgcctaacaagcactatttcgaaatcgacctgtcctggcacaagggactgcagaatactggtaaaaacgcagaggtgttcgcccctcagagtgatcccaatggtctgatcaaatgcacagtggggagatcctctctgaagagcaagctg